(19) United States
(12) Patent Application Publication
(10) Pub. No.: US 2022/0381671 A1
(43) Pub. Date: Dec. 1, 2022

(54) INFORMATION PROCESSING SYSTEM AND INFORMATION PROCESSING APPARATUS

(71) Applicant: Sony Group Corporation, Tokyo (JP)

(72) Inventor: Yuji Nishimaki, Tokyo (JP)

(73) Assignee: Sony Group Corporation, Tokyo (JP)

(21) Appl. No.: 17/776,566

(22) PCT Filed: Nov. 13, 2020
(86) PCT No.: PCT/JP2020/042404

§ 371 Date: May 12, 2022

(30) Foreign Application Priority Data
Nov. 20, 2019 (JP) 2019-209936

(51) Int. Cl. G01N 15/14

(52) U.S. Cl. CPC G01N 15/1429; G01N 2015/1402

(57) ABSTRACT

An information processing system and an information processing apparatus capable of reducing an amount of data to be transferred from a local environment to a cloud environment are provided. The information processing system according to the embodiment includes a first information processing apparatus (100) and a second information processing apparatus (200), the first information processing apparatus includes a first processing unit (103) configured to generate compressed data by irradiating a measurement target dyed with a plurality of fluorescent dyes with light and performing compression processing on measurement data measured by the irradiation, using reference data for each of the fluorescent dyes used for dyeing the measurement target; and a transmission unit (104) configured to transmit the compressed data to the second information processing apparatus, and the second information processing apparatus includes a second processing unit (203) configured to generate restored data by performing restoration processing using the reference data and the compressed data received from the first information processing apparatus.

### FIELD

The present disclosure relates to an information processing system and an information processing apparatus.

### BACKGROUND

In the fields of medicine, biochemistry, and the like, a flow cytometer is sometimes used to quickly measure property of a large amount of particles. The flow cytometer, which is a measurement apparatus using an analytical method called flow cytometry, irradiates particles such as cells flowing through a flow cell with light and detects fluorescence emitted from the particles.

The following Patent Literature 1 discloses, in fluorescence detection of a flow cytometer (microparticle measurement apparatus), detecting intensity of light in a continuous wavelength region as a fluorescence spectrum. In the microparticle measurement apparatus disclosed in Patent Literature 1, by using a spectroscopic element such as a prism or a grating, fluorescence emitted from particles such as cells dyed using a plurality of fluorescent dyes is dispersed, and the dispersed fluorescence is detected by a light receiving element array in which a plurality of light receiving elements having different detection wavelength regions is arranged. By collecting detection values of the respective light receiving elements constituting the light receiving element array, a fluorescence spectrum of a measurement target such as cells can be measured.

Such a flow cytometer is called a spectral flow cytometer. The spectral flow cytometer has an advantage that information on fluorescence can be fully utilized as analysis information, as compared with a filter method in which fluorescence is separated and detected for each wavelength region using an optical filter.

Further, for example, the following Patent Literatures 2 to 5 disclose a method in which a fluorescence spectrum (measurement spectrum) obtained by a spectral flow cytometer is approximated by a linear sum of reference data (single dyeing spectrum) representing standard fluorescence wavelength distribution for each fluorescent dye to obtain measurement data representing a measurement result for each fluorescent dye. Such a method is called spectral unmixing (hereinafter, simply referred to as “unmixing”).

### CITATION LIST

### Patent Literature

Patent Literature 1: JP 5772425 B2
Patent Literature 2: JP 5985140 B2
Patent Literature 3: JP 5540952 B2
Patent Literature 4: JP 5601098 B2
Patent Literature 5: JP 5834584 B2

### SUMMARY

### Technical Problem

Use of a spectral flow cytometer is advantageous because a measurement spectrum in which spectra of a plurality of fluorescent dyes are mixed, and measurement data representing a measurement result for each fluorescent dye can be acquired, and thus, analysis of a measurement target can be finely performed using both of them. However, in order to perform such analysis in a local environment, it is necessary to secure sufficient calculation resources in the local environment.

It is therefore considered to transfer data obtained in the local environment to a cloud environment and analyze the measurement target in the cloud environment. Use of an analysis application in the cloud environment enables detailed analysis of the measurement target to be easily performed by utilizing sufficient calculation resources of the cloud environment and enables data sharing, or the like, to be easily performed, which improves user-friendliness. However, in this case, if an amount of data to be transferred from the local environment to the cloud environment is large, a data transfer period and a communication band to be used for data transfer increase, and storage cost required for storing data in the cloud environment also increases. It is therefore desired to reduce the amount of data to be transferred from the local environment to the cloud environment.

### Solution to Problem

In accordance with the present disclosure, an information processing system comprises: a first information processing apparatus; and a second information processing apparatus, the first information processing apparatus including: a first processing unit configured to generate compressed data by irradiating a measurement target dyed with a plurality of fluorescent dyes with light and performing compression processing on measurement data measured by the irradiation, using reference data for each of the fluorescent dyes used for dyeing the measurement target; and a transmission unit configured to transmit the compressed data to the second information processing apparatus, the second information processing apparatus including: a second processing unit configured to generate restored data by performing restoration processing using the reference data and the compressed data received from the first information processing apparatus.

Furthermore, in accordance with the present disclosure, an information processing apparatus comprises: a first processing unit configured to generate compressed data by performing compression processing on measurement data measured by irradiating a measurement target dyed using a plurality of fluorescent dyes with light, using reference data for each of the fluorescent dyes used for dyeing the measurement target; and a second processing unit configured to generate restored data by performing restoration processing using the reference data and the compressed data.

### BRIEF DESCRIPTION OF DRAWINGS

FIG. 1 is a view illustrating general data flow in a case where an analysis application is used in a cloud environment.

FIG. 2 is a view illustrating an example of data flow in a case where unmixing is performed in a local environment.

FIG. 3 is a view illustrating an example of data flow according to the present disclosure.

FIG. 4A is a view for explaining data reproducibility by inverse transformation of unmixing.

FIG. 4B is a view for explaining data reproducibility by inverse transformation of unmixing.

FIG. 5 is a block diagram illustrating a configuration example of an information processing system according to a first embodiment.

FIG. 6 is a view illustrating a schematic configuration example of a flow cytometer.

FIG. 7 is a view explaining outline of unmixing.

FIG. 8 is a flowchart illustrating flow of a series of processing to be performed in an information processing system according to the first embodiment.

FIG. 9 is a view illustrating an example of data flow according to a second embodiment.

FIG. 10 is a block diagram illustrating a configuration example of an information processing system according to the second embodiment.

FIG. 11 is a flowchart illustrating flow of a series of processing to be performed in an information processing system according to the second embodiment.

FIG. 12 is a view illustrating an example of data flow according to a third embodiment.

FIG. 13 is a block diagram illustrating a configuration example of an information processing system according to the third embodiment.

FIG. 14 is a flowchart illustrating flow of a series of processing to be performed in an information processing system according to the third embodiment.

FIG. 15 is a block diagram illustrating a configuration example of an information processing system according to a fourth embodiment.

FIG. 16 is a flowchart explaining characteristic processing to be performed in the information processing system according to the fourth embodiment.

FIG. 17 is a block diagram explaining a modification.

FIG. 18 is a view illustrating a schematic configuration example of a fluorescence imaging apparatus.

FIG. 19 is a view illustrating an example of a hardware configuration of the information processing apparatus.

### DESCRIPTION OF EMBODIMENTS

Favorable embodiments of the present disclosure will be described in detail with reference to the appended drawings. In this specification and the drawings, components having substantially the same functional configuration are denoted by the same reference numerals, and redundant description is omitted.

Note that the description will be given in the following order.

1. Outline of the present disclosure

2. First Embodiment

3. Second Embodiment

4. Third Embodiment

5. Fourth Embodiment

6. Fifth Embodiment

7. Hardware configuration example

8. Supplementary description

### 1. Outline of Present Disclosure

For example, in order to improve user-friendliness of analysis of a measurement target using a spectral flow cytometer, it has been studied to use an analysis application in a cloud environment. While user-friendliness is improved by use of the analysis application in the cloud environment, it is necessary to transfer data required for analysis processing from a local environment to the cloud environment and store the data in the cloud environment. Here, if an amount of data to be transferred from the local environment to the cloud environment can be reduced, a data transfer period and a data communication band can be reduced. Further, storage cost for storage in the cloud environment can be reduced.

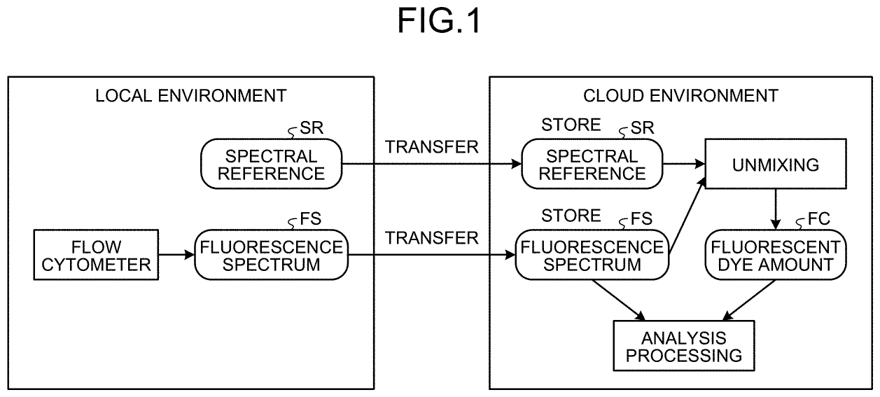

General data flow in a case where the analysis application is used in the cloud environment is illustrated in FIG. 1. In analysis of a measurement target using the spectral flow cytometer, measurement data (hereinafter, referred to as a “fluorescence spectrum”) FS to be measured by irradiating the measurement target dyed using a plurality of fluorescent dyes with light and compressed data (hereinafter, referred to as a “fluorescent dye amount”) FC obtained by performing data compression processing on the fluorescence spectrum FS using reference data (hereinafter, referred to as a “spectral reference”) SR representing standard fluorescence wavelength distribution for each of the fluorescent dyes used for dyeing the measurement target are used.

The data compression processing for generating the fluorescent dye amount FC may be lossy compression, linear processing, or nonlinear processing. The nonlinear processing may include, for example, dimension compression processing, clustering processing, grouping processing, and the like. Further, the linear processing may include, for example, processing of generating fluorescence information for each fluorescent dye from spectrum information of light of biogenic particles by performing fluorescence separation. In the present description, such data compression processing is referred to as unmixing.

In a case where the analysis application is used in the cloud environment, it is generally conceivable to perform this unmixing in the cloud environment. Thus, in this case, as illustrated in FIG. 1, the fluorescence spectrum FS acquired from the spectral flow cytometer and the spectral reference SR to be used for unmixing need to be transferred from the local environment to the cloud environment and stored in the cloud environment.

A problem here is that a data size of the fluorescence spectrum FS is very large. Thus, a data transfer period from the local environment to the cloud environment becomes long, and there are problems such as a problem that a large amount of bandwidth is used for data transfer and a problem that storage cost for storing data in the cloud environment becomes high.

Several approaches are conceivable for such problems. For example, there is a method in which the fluorescence spectrum FS is transferred after a data amount is reduced by lossless compression (or deletion of bits which are not used) before the fluorescence spectrum FS is transferred to the cloud environment. However, it is desirable to restore the same data as that before compression through lossless compression, which limits a compression rate and degrades efficiency.

Further, there can be a method in which the fluorescence spectrum FS is lossy-compressed before the fluorescence spectrum FS is transferred to the cloud environment to reduce an amount of data and then transferred. In this method, a high compression rate of data can be expected as compared with lossless compression. However, if the lossy-compressed fluorescence spectrum FS is restored in the cloud environment, an error occurs. The error occurring here increases due to unmixing to be performed in the cloud environment, which causes a problem that an error in the fluorescent dye amount FC to be generated in the cloud environment becomes very large. This is because repetitive product-sum operation is performed on the fluorescence spectrum FS when the fluorescent dye amount FC is generated through unmixing. By this product-sum operation, the error of the fluorescence spectrum FS increases and propagates to the fluorescent dye amount FC.

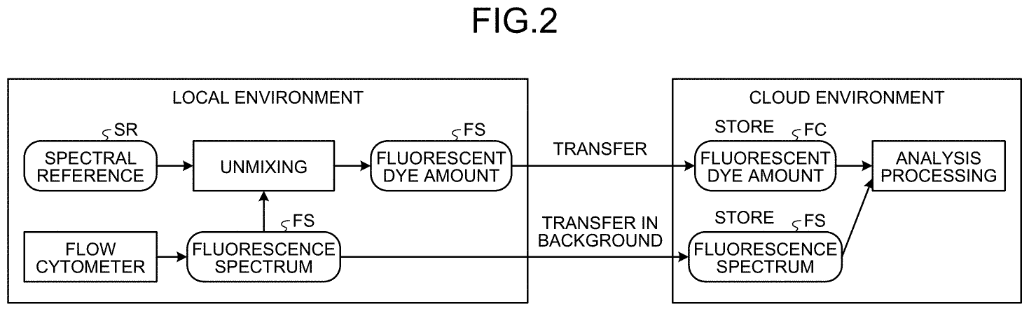

Further, there can also be a method in which unmixing is performed in the local environment to generate the fluorescent dye amount FC for each fluorescent dye, the fluorescent dye amount FC is transferred from the local environment to the cloud environment, and the fluorescence spectrum FS is transmitted from the local environment to the cloud environment in the background. Data flow in this case is illustrated in FIG. 2.

The fluorescent dye amount FC is lower in dimension than the fluorescence spectrum FS and has a characteristic that a data size is sufficiently smaller than that of the fluorescence spectrum FS. Further, a period required for unmixing is sufficiently shorter than a period required for data transfer of the fluorescence spectrum FS. Thus, by generating the fluorescent dye amount FC in the local environment and transferring the generated fluorescent dye amount FC to the cloud environment, the fluorescent dye amount FC can be used for analysis earlier than in a case where unmixing is performed in the cloud environment. However, not only the fluorescent dye amount FC but also the fluorescence spectrum FS is used in the analysis processing as described above, and thus, the user needs to wait for the fluorescence spectrum FS to be transferred to the cloud environment in the background. Further, in this method, it is necessary to store both the fluorescence spectrum FS having a large data size and the fluorescent dye amount FC in the cloud environment, which cannot solve the problem that the storage cost for storing data in the cloud environment increases.

Thus, the present disclosure uses a method in which the fluorescence spectrum FS is restored from the fluorescent dye amount FC and the spectral reference SR in the cloud environment using inverse transformation of unmixing. This eliminates the need to transfer the fluorescence spectrum FS from the local environment to the cloud environment and store the fluorescence spectrum FS in the cloud environment, which leads to reduction in a data transfer period, a communication band, and storage cost.

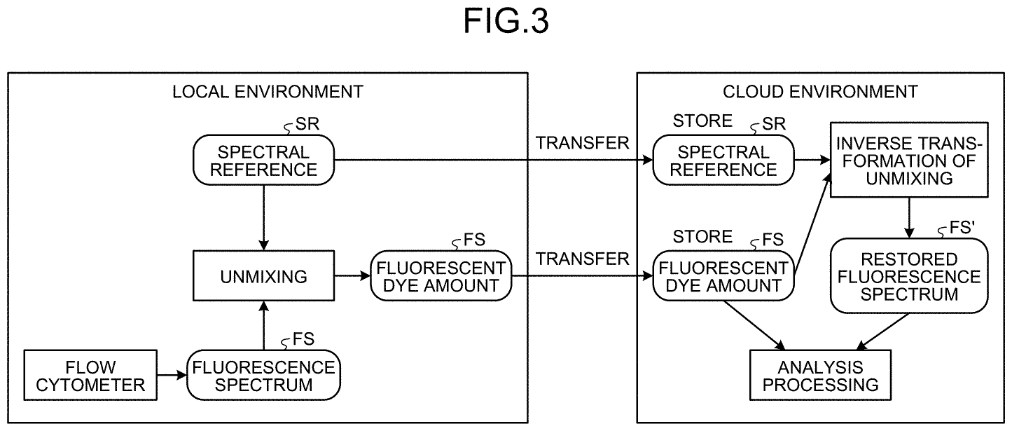

An example of data flow of the present disclosure is illustrated in FIG. 3. In the present disclosure, as illustrated in FIG. 3, the fluorescent dye amount FC is generated by unmixing the fluorescence spectrum FS acquired from the spectral flow cytometer in the local environment. Then, the fluorescent dye amount FC and the spectral reference SR used for unmixing are transferred from the local environment to the cloud environment. Thereafter, in the cloud environment, inverse transformation of unmixing performed in the local environment is performed using the fluorescent dye amount FC and the spectral reference SR transferred from the local environment to restore the fluorescence spectrum FS. The restored fluorescence spectrum FS is referred to as a restored fluorescence spectrum FS′.

The restored fluorescence spectrum FS′ obtained by inverse transformation of unmixing does not accurately reproduce the original fluorescence spectrum FS, but is data sufficiently close to the original fluorescence spectrum FS. Thus, by performing analysis processing using the restored fluorescence spectrum FS′ and the fluorescent dye amount FC, it is possible to finely analyze the measurement target as in a case of using the original fluorescence spectrum FS and the fluorescent dye amount FC.

Here, reproducibility of data by inverse transformation of unmixing will be considered. An expression of unmixing is indicated below. Here, S represents a spectral reference SR, xᵢ (where i is 1 to n) represents a value of the fluorescent dye amount FC for each fluorescent dye, n represents the number of fluorescent dyes, yᵢ (where i is 1 to m) represents a value of the fluorescence spectrum FS for each detection channel set for each frequency region, and m represents the number of detection channels. Here, an example where unmixing is performed by a weighted least square method will be described, but unmixing may be performed using other methods such as a least square method.

$$[ \begin{matrix}{x}_{1} \\ ⋮ \\ {x}_{n}\end{matrix} ] = {( [ {S}^{T} ] [ L ] [ S ] )}^{- 1} [ {S}^{T} ] [ L ] [ \begin{matrix}{y}_{1} \\ ⋮ \\ {y}_{m}\end{matrix} ]$$

$$L = [ \begin{matrix}{λ}_{1} & 0 & 0 \\ 0 & ⋱ & 0 \\ 0 & 0 & {λ}_{m}\end{matrix} ] , {λ}_{i} = \frac{1}{max ( {y}_{i} , 0 ) + offset} , n < m$$

Here, a case will be considered as a simple example where the number of fluorescent dyes is two and the number of detection channels is three. In this case, relationship between the fluorescent dye amount FC and the fluorescence spectrum FS can be expressed using three expressions: S₁₁·x₁+S₁₂·x₂=y₁, S₂₁·x₁+S₂₂·x₂=y₂, and S₃₁·x₁+S₃₂·x₂=y₃. There are three solutions (x₁, x₂) to these equations. Processing of obtaining one most probable solution from these solutions is unmixing processing. In other words, unmixing means obtaining x₁ and x₂ which are the closest to the following expression.

Σₙ₌₀³(yₙ−Sₙ₁·x₁−Sₙ₂·x₂)=0

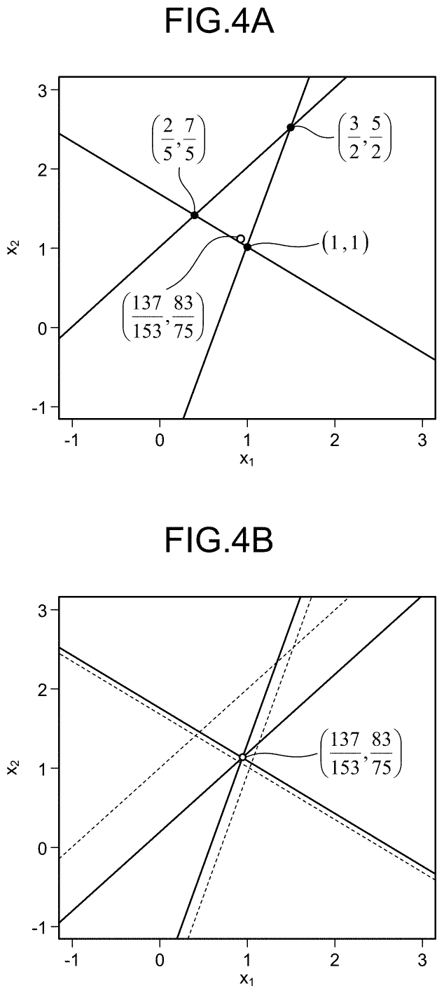

For example, by substituting appropriate numerical values for S and y and replacing the above three expressions with three expressions of 2x₁+3x₂=5, 3x₁−x₂=2, and −x₁+x₂=1, three solutions of (2/5, 7/5), (1,1), and (3/2, 5/2) can be obtained, as illustrated in FIG. 4A. In this case, one solution (137/153, 83/75) can be obtained as a result of unmixing.

If (137/153, 83/75) is obtained through unmixing, a value close to y can be obtained from S and x. In other words, if x₁=137/153 and x₂=83/75 are respectively substituted into the three expressions of 2x₁+3x₂=5, 3x₁−x₂=2, and −x₁+x₂=1, y₁=5.14667, y₂=1.633333, and y₃=0.1933333 are obtained, which are respectively close to the values of y in the original three expressions. This processing corresponds to inverse transformation of unmixing.

The three expressions (2x₁+3x₂=5.14667, 3x₁−x₂=1.633333, −x₁+x₂=0.1933333) restored through inverse transformation of unmixing are illustrated in FIG. 4B together with the original three equations (2x₁+3x₂=5, 3x₁−x₂=2, −x₁+x₂=1). A solid line in the drawing indicates an expression restored through inverse transformation of unmixing, and a broken line indicates an original expression. As can be seen from FIG. 4B, the original data cannot be completely reproduced by inverse transformation of unmixing, but data having a close value can be restored.

As described above, according to the method of the present disclosure, the fluorescence spectrum FS is restored in the cloud environment by inverse transformation of unmixing using the fluorescent dye amount FC and the spectral reference SR transferred from the local environment to the cloud environment, so that it is not necessary to transfer the fluorescence spectrum FS having a large data size from the local environment to the cloud environment. It is therefore possible to reduce an amount of data to be transferred from the local environment to the cloud environment. Further, the fluorescence spectrum FS can be restored by performing inverse transformation of unmixing at the time of executing analysis processing, so that it is not necessary to always store the fluorescence spectrum FS in the cloud environment. It is therefore possible to reduce an amount of data to be stored in the cloud environment, so that it is possible to reduce storage cost.

### 2. First Embodiment

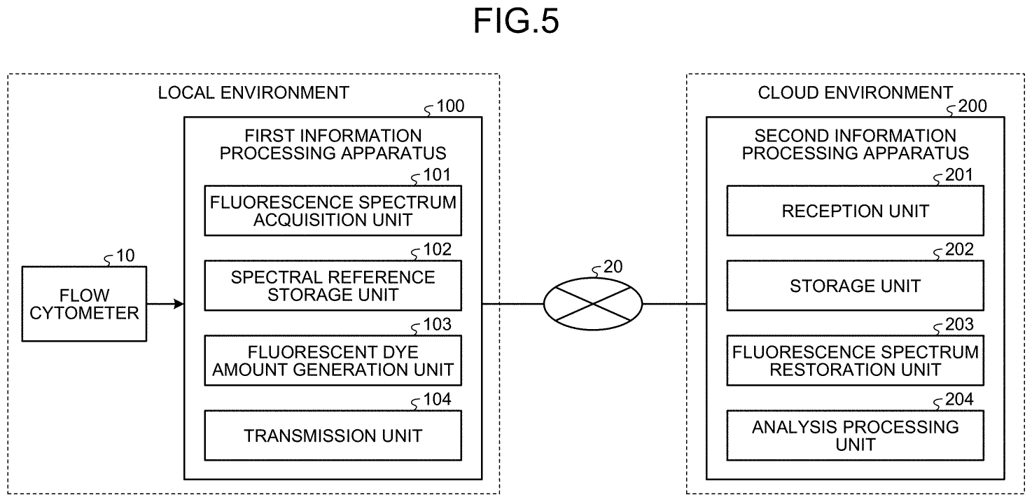

FIG. 5 is a block diagram illustrating a configuration example of an information processing system according to a first embodiment. As illustrated in FIG. 5, the information processing system according to the present embodiment includes a flow cytometer 10 and a first information processing apparatus 100 provided in the local environment, and a second information processing apparatus 200 provided in the cloud environment. The first information processing apparatus 100 provided in the local environment and the second information processing apparatus 200 provided in the cloud environment are connected via a network 20. For example, the network 20 may include a public network such as the Internet, a telephone network, or a satellite communication network, various local area networks (LANs) including Ethernet (registered trademark), a wide area network (WAN), and the like.

The flow cytometer 10 measures the fluorescence spectrum FS (measurement data) by irradiating the measurement target dyed using a plurality of fluorescent dyes with light. The measurement target may be a biogenic particle such as a cell, a tissue, a microorganism and a bio-related particle. For example, the cell may be an animal cell (such as, for example, a blood cell), a plant cell, or the like. For example, the tissue may be a tissue collected from a human body or the like, or may be part (including a tissue cell) of the tissue instead of the entire tissue. For example, the microorganism may be a bacterium such as Escherichia coli, a virus such as a tobacco mosaic virus, a fungus such as a yeast, or the like. The bio-related particle may be a particle constituting a cell such as a chromosome, a liposome, a mitochondrion, or various organelles (organelles). Note that the bio-related particle may include a bio-related polymer such as a nucleic acid, protein, lipid, and a sugar chain, and combinations thereof. These biogenic particle may have either a spherical shape or a non-spherical shape and is not particularly limited in terms of size and mass.

The measurement target may be an industrially synthesized particle such as a latex particle, a gel particle, and an industrial particle. For example, the industrially synthesized particle may be a particle synthesized with an organic resin material such as polystyrene and polymethyl methacrylate, an inorganic material such as glass, silica and a magnetic body, or a metal such as colloidal gold and aluminum. The industrially synthesized particle may also have either a spherical shape or a non-spherical shape and is not particularly limited in size and mass in a similar manner.

The measurement target is dyed (labeled) using a plurality of fluorescent dyes prior to measurement of the fluorescence spectrum FS. The measurement target may be labeled with the fluorescent dyes using a known method. Specifically, in a case where the measurement target is a cell, the measurement target cell can be labeled with the fluorescent dyes by mixing a fluorescently labeled antibody that selectively binds to an antigen present on the cell surface with the measurement target cell and binding the fluorescently labeled antibody to the antigen on the cell surface. Alternatively, it is also possible to label a measurement target cell with the fluorescent dyes by mixing a fluorescent dye that is selectively taken up for a specific cell with the measurement target cell.

The fluorescently labeled antibody is an antibody to which the fluorescent dyes are caused to bind as a label. The fluorescently labeled antibody may be an antibody to which the fluorescent dyes are caused to directly bind. Alternatively, the fluorescently labeled antibody may be an antibody obtained by binding the fluorescent dyes to which avidin is caused to bind to a biotin-labeled antibody through avidin-biotin reaction. Note that as the antibody, either a polyclonal antibody or a monoclonal antibody can be used.

The fluorescent dyes for labeling a cell are not particularly limited, and known dyes to be used for dyeing a cell, or the like, can be used. For example, as fluorescent dyes, phycoerythrin (PE), fluorescein isothiocyanate (FITC), PE-Cy5, PE-Cy7, PE-Texas Red (registered trademark), allophycocyanin (APC), APC-Cy7, ethidium bromide, propidium iodide, Hoechst (registered trademark) 33258, Hoechst (registered trademark) 33342, DAPI (4′,6-diamidino-2-phenylindole), acridineorange, chromomycin, mithramycin, olivomycin, pyronin Y, thiazole orange, rhodamine 101, isothiocyanate, BCECF, BCECF-AM, C.SNARF-1, C.SNARF-1 AMA, aequorin, Indo-1, Indo-1-AM, Fluo-3, Fluo-3-AM, Fura-2, Fura-2-AM, oxonol, Texas Red (registered trademark), Rhodamine 123, 10-N-nony-acridine orange, fluorescein, fluorescein diacetate, carboxyfluorescein, carboxyfluorescein diacetate, carboxydichlorofluorescein, carboxydichlorofluorescein diacetate, and the like, can be used. Further, derivatives of the above-described fluorescent dyes, and the like, can also be used.

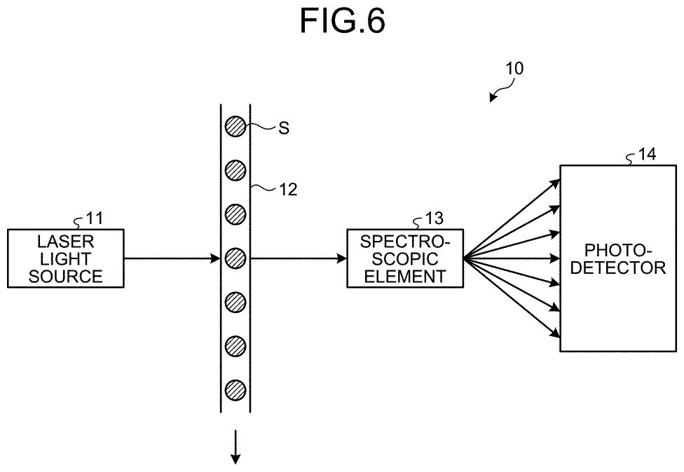

A schematic configuration example of the flow cytometer 10 is illustrated in FIG. 6. As illustrated in FIG. 6, the flow cytometer 10 includes a laser light source 11, a flow cell 12, a spectroscopic element 13, and a photodetector 14.

The laser light source 11 emits laser light having a wavelength capable of exciting the fluorescent dyes used for dyeing the measurement target (sample) S. Although only one laser light source 11 is illustrated in FIG. 6, a plurality of laser light sources 11 may be provided. As the laser light source 11, for example, a semiconductor laser light source that emits laser light having a predetermined wavelength can be used. The laser light emitted from the laser light source 11 may be pulsed light or continuous light.

The flow cell 12 is a flow path that allows the measurement target S such as a cell to flow while being aligned in one direction. Specifically, the flow cell 12 causes a sheath liquid enclosing the measurement target S such as a cell to flow at high speed as laminar flow to align and flow the measurement target S such as a cell in one direction.

The spectroscopic element 13 is an optical element that disperses fluorescence emitted from the measurement target S into a spectrum of a continuous wavelength by being irradiated with the laser light from the laser light source 11. As the spectroscopic element 13, for example, a prism, a grating, or the like, can be used.

The photodetector 14 includes a light receiving element array that detects fluorescence which is generated from the measurement target S irradiated with the laser light and which is dispersed by the spectroscopic element 13. The light receiving element array has, for example, a configuration in which a plurality of independent detection channels having different wavelength regions of light to be detected is arranged. Specifically, the light receiving element array is constituted by, for example, arranging light receiving elements such as a plurality of photo multiplier tubes (PMT) or photodiodes having different wavelength regions to be detected in one dimension along a spectral direction by the spectroscopic element 13. The number of light receiving elements constituting the light receiving element array, that is, the number of detection channels is set to be larger than the number of fluorescent dyes to be used for dyeing the measurement target S.

In the flow cytometer 10 constituted as described above, fluorescence is emitted from the measurement target S by the measurement target S flowing through the flow cell 12 being irradiated with the laser light from the laser light source 11. The fluorescence emitted from the measurement target S is dispersed into a continuous spectrum by the spectroscopic element 13 and is received (detected) by the plurality of light receiving elements constituting the light receiving element array of the photodetector 14. This makes it possible to measure the fluorescence spectrum FS of the measurement target S dyed using a plurality of fluorescent dyes.

As illustrated in FIG. 5, the first information processing apparatus 100 provided in the local environment and connected to the flow cytometer 10 includes a fluorescence spectrum acquisition unit 101, a spectral reference storage unit 102, a fluorescent dye amount generation unit 103 (an example of a “first processing unit”), and a transmission unit 104. Note that part or all of the functions of the first information processing apparatus 100 may be implemented inside the flow cytometer 10. In other words, at least part of the first information processing apparatus 100 may be integrated with the flow cytometer 10.

The fluorescence spectrum acquisition unit 101 acquires a fluorescence spectrum FS (measurement data) which is measurement data by the flow cytometer 10.

The spectral reference storage unit 102 stores a spectral reference SR (reference data) representing standard fluorescence wavelength distribution for each fluorescent dye. The spectral reference storage unit 102 stores spectral references SR of various fluorescent dyes that can be used in fluorescence detection by the flow cytometer 10, for example, in a library format. Note that the spectral reference storage unit 102 may be provided in a server apparatus, or the like, outside the first information processing apparatus 100.

The fluorescent dye amount generation unit 103 unmixes the fluorescence spectrum FS acquired by the fluorescence spectrum acquisition unit 101 using the spectral reference SR corresponding to each fluorescent dye used for dyeing the measurement target S among the spectral references SR stored in the spectral reference storage unit 102 to generate the fluorescent dye amount FC representing the measurement result for each fluorescent dye used for dyeing the measurement target S.

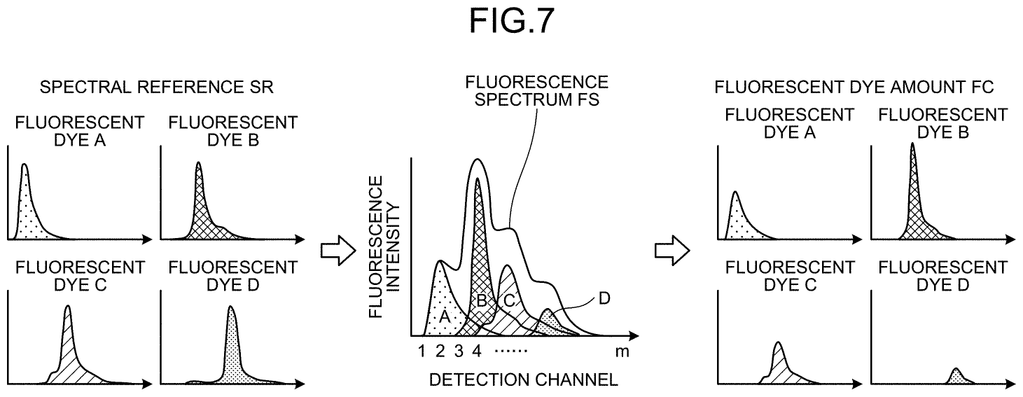

FIG. 7 is a view explaining outline of unmixing to be performed by the fluorescent dye amount generation unit 103. As illustrated in FIG. 7, the fluorescence spectrum FS of the measurement target S to be measured by the flow cytometer 10 is a mixture of spectra of a plurality of fluorescent dyes used for dyeing the measurement target S. Unmixing is processing of separating the fluorescence spectrum FS in which the spectra of the plurality of fluorescent dyes used for dyeing the measurement target S are mixed into spectra for each fluorescent dye using the spectral reference SR corresponding to each fluorescent dye and obtaining the fluorescent dye amount FC representing the measurement result for each fluorescent dye in this manner.

Specifically, it is assumed that the fluorescence spectrum FS of the measurement target S dyed with a plurality of fluorescent dyes is expressed by a linear sum of spectral references SR corresponding to the respective fluorescent dyes used for dyeing the measurement targets S, and the fluorescent dye amount FC which is a measurement result for each fluorescent dye can be derived by superimposing the spectral reference SR corresponding to each fluorescent dye and fitting the spectral reference SR to the fluorescence spectrum FS to obtain a coupling coefficient for each fluorescent dye of a linear coupling. A calculation method such as a weighted least square method or a least square method can be used in fitting. Note that a specific example of such a calculation method is described in detail in Patent Literatures 2 to 5, and the like, and thus, detailed description thereof will be omitted here.

The transmission unit 104 transmits the fluorescent dye amount FC generated by the fluorescent dye amount generation unit 103, that is, the fluorescent dye amount FC representing the measurement result for each fluorescent dye used for dyeing the measurement target S, and the spectral references SR used for unmixing at the fluorescent dye amount generation unit 103, that is, the spectral references SR corresponding to the respective fluorescent dyes used for dyeing the measurement target S to the second information processing apparatus 200 via the network 20. Here, it should be noted that the transmission unit 104 does not transmit the fluorescence spectrum FS, which is measurement data by the flow cytometer 10, to the second information processing apparatus 200. In other words, in the information processing system according to the present embodiment, the fluorescence spectrum FS having a large data size is not transmitted from the first information processing apparatus 100 to the second information processing apparatus 200, and only the fluorescent dye amount FC and the spectral reference SR having a small data size are transmitted from the first information processing apparatus 100 to the second information processing apparatus 200.

On the other hand, as illustrated in FIG. 5, the second information processing apparatus 200 provided in the cloud environment includes a reception unit 201, a storage unit 202, a fluorescence spectrum restoration unit 203 (an example of a “second processing unit”), and an analysis processing unit 204.

The reception unit 201 receives the fluorescent dye amount FC and the spectral references SR transmitted from the first information processing apparatus 100 via the network 20.

The storage unit 202 stores the fluorescent dye amount FC and the spectral references SR received by the reception unit 201 from the first information processing apparatus 100.

The fluorescence spectrum restoration unit 203 restores the fluorescence spectrum FS, which is the measurement data by the flow cytometer 10, by performing inverse transformation of unmixing performed when the fluorescent dye amount generation unit 103 of the first information processing apparatus 100 generates the fluorescent dye amount FC, using the fluorescent dye amount FC stored in the storage unit 202 and the spectral references SR and generates the restored fluorescence spectrum FS′. As described above, the restored fluorescence spectrum FS′ does not accurately reproduce the original fluorescence spectrum FS, but is data sufficiently close to the original fluorescence spectrum FS. The restored fluorescence spectrum FS′ is preferably generated (the fluorescence spectrum FS is preferably restored) by the fluorescence spectrum restoration unit 203 immediately before the analysis processing by the analysis processing unit 204. As a result, the generated restored fluorescence spectrum FS′ can be used as is in the analysis processing by the analysis processing unit 204 without being permanently stored.

The analysis processing unit 204 analyzes the measurement target S using the fluorescent dye amount FC stored in the storage unit 202 and the restored fluorescence spectrum FS′ generated by the fluorescence spectrum restoration unit 203. This analysis processing may include, for example, clustering processing on the measurement target S. By the clustering processing, the measurement target S such as a cell can be classified into a plurality of groups obtained through external isolation and internal cohesion. Algorithm of the clustering processing is not particularly limited, and known clustering algorithm can be used. For example, the analysis processing unit 204 may perform clustering processing using algorithm such as k-means that can specify the number of clusters or may perform clustering processing using algorithm such as flowsom that automatically determines the number of clusters.

Further, the analysis processing unit 204 may present a result of the analysis processing such as clustering processing to the user. In a case where the result of the clustering processing is presented to the user, the analysis processing unit 204 may display the result of the clustering processing in, for example, a table format or a minimum spanning tree format. In addition to the clustering processing, the analysis processing unit 204 can perform various kinds of analysis processing using the fluorescent dye amount FC and the restored fluorescence spectrum FS′ in accordance with operation of the user and can present the result to the user.

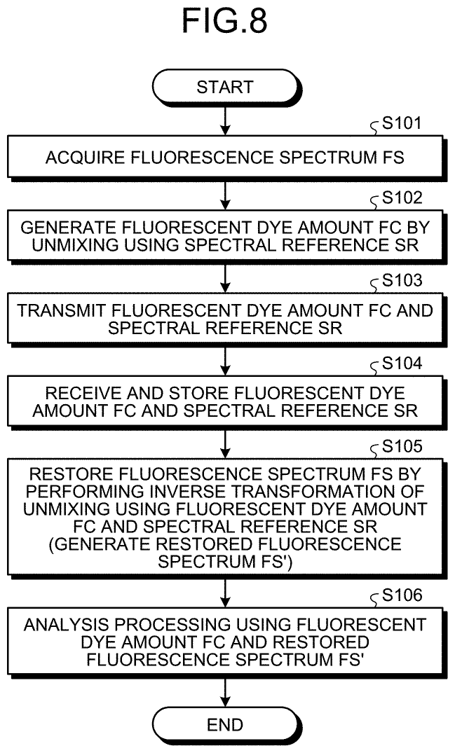

FIG. 8 is a flowchart illustrating flow of a series of processing to be performed in an information processing system according to the first embodiment. Hereinafter, outline of operation of the information processing system according to the present embodiment will be described along the flowchart of FIG. 8.

First, if the fluorescence spectrum FS of the measurement target S dyed using a plurality of fluorescent dyes is measured by the flow cytometer 10, the fluorescence spectrum acquisition unit 101 of the first information processing apparatus 100 acquires the fluorescence spectrum FS (Step S101).

Next, the fluorescent dye amount generation unit 103 of the first information processing apparatus 100 unmixes the fluorescence spectrum FS acquired by the fluorescence spectrum acquisition unit 101 in Step S101 using the spectral reference SR corresponding to each fluorescent dye used for dyeing the measurement target S among the spectral references SR stored in the spectral reference storage unit 102 to generate the fluorescent dye amount FC representing the measurement result for each fluorescent dye (Step S102).

Next, the transmission unit 104 of the first information processing apparatus 100 transmits the fluorescent dye amount FC generated by the fluorescent dye amount generation unit 103 in Step S102 and the spectral references SR used for unmixing to the second information processing apparatus 200 via the network 20 (Step S103).

Next, the reception unit 201 of the second information processing apparatus 200 receives the fluorescent dye amount FC and the spectral references SR transmitted from the first information processing apparatus 100 via the network 20 and stores them in the storage unit 202 (Step S104).

Thereafter, the fluorescence spectrum restoration unit 203 of the second information processing apparatus 200 restores the fluorescence spectrum FS by performing inverse transformation of unmixing performed by the fluorescent dye amount generation unit 103 of the first information processing apparatus 100 in Step S102, using the fluorescent dye amount FC stored in the storage unit 202 and the spectral references SR to generate the restored fluorescence spectrum FS′ (Step S105).

Then, the analysis processing unit 204 of the second information processing apparatus 200 performs analysis processing on the measurement target S using the fluorescent dye amount FC stored in the storage unit 202 and the restored fluorescence spectrum FS′ generated by the fluorescence spectrum restoration unit 203 in Step S105 (Step S106), and a series of processing ends.

As described above, according to the information processing system according to the present embodiment, as a result of the fluorescent dye amount FC and the spectral references SR being transferred from the first information processing apparatus 100 in the local environment to the second information processing apparatus 200 in the cloud environment, and inverse transformation of unmixing being performed at the second information processing apparatus 200 in the cloud environment using the fluorescent dye amount FC and the spectral references SR, the restored fluorescence spectrum FS′ is generated by the fluorescence spectrum FS being restored. This enables analysis processing to be performed using the fluorescent dye amount FC and the restored fluorescence spectrum FS′ at the second information processing apparatus 200 in the cloud environment without the fluorescence spectrum FS having a large data size being transferred from the first information processing apparatus 100 in the local environment to the second information processing apparatus 200 in the cloud environment, which makes it possible to reduce an amount of data to be transferred from the local environment to the cloud environment and reduce an amount of data to be stored in the cloud environment, so that it is possible to reduce storage cost.

### 3. Second Embodiment

As described above, the restored fluorescence spectrum FS′ generated by restoring the fluorescence spectrum FS by inverse transformation of unmixing is data sufficiently close to the original fluorescence spectrum FS, but does not completely reproduce the original fluorescence spectrum FS. This is because unmixing performed on the original fluorescence spectrum FS has a characteristic of deleting information other than the spectrum information of the spectral references SR used for the unmixing.

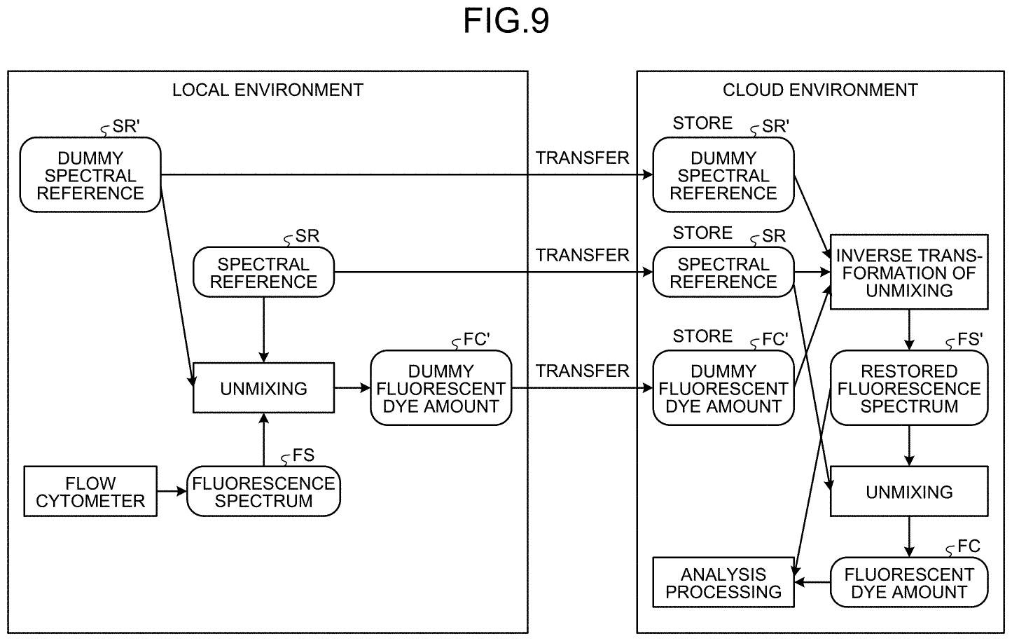

In the present embodiment, an example of a method for increasing a reproduction rate of the fluorescence spectrum FS will be described. An example of data flow of the present embodiment is illustrated in FIG. 9. In the present embodiment, as illustrated in FIG. 9, when unmixing is performed on the fluorescence spectrum FS in the local environment, in addition to the spectral references SR corresponding to the fluorescent dyes used for dyeing the measurement target S, a dummy spectral reference SR′, which is dummy data independent of the fluorescent dyes used for dyeing the measurement target S, is used. The dummy spectral reference SR′ includes spectral information not included in the spectral references SR corresponding to the fluorescent dyes used for dyeing the measurement target S, and thus, by adding the dummy spectral reference SR′, it is possible to leave spectral information that is originally deleted by unmixing, so that it is possible to improve reproducibility of the restored fluorescence spectrum FS′ generated by inverse transformation of unmixing.

If unmixing is performed on the fluorescence spectrum FS using the dummy spectral reference SR′ in addition to the spectral references SR, a dummy fluorescent dye amount FC′ in which dummy data is added to the original fluorescent dye amount FC is generated. In the present embodiment, the dummy fluorescent dye amount FC′, the spectral references SR, and the dummy spectral reference SR′ are transferred from the local environment to the cloud environment. Then, by inverse transformation of unmixing being performed in the cloud environment using the dummy fluorescent dye amount FC′, the spectral references SR and the dummy spectral reference SR′, the restored fluorescence spectrum FS′ with high reproducibility is generated.

In addition, in the present embodiment, the dummy fluorescent dye amount FC′ is transferred from the local environment to the cloud environment instead of the fluorescent dye amount FC, and thus, it is necessary to generate the fluorescent dye amount FC in the cloud environment. Thus, in the cloud environment, unmixing is performed on the restored fluorescence spectrum FS′ using the spectral references SR to generate the fluorescent dye amount FC. Then, the measurement target S is subjected to analysis processing using the fluorescent dye amount FC and the restored fluorescence spectrum FS′.

According to the method of the present embodiment, as an amount of data of the dummy spectral reference SR′ increases, an amount of data of the dummy fluorescent dye amount FC′ increases, and a reproduction rate of the restored fluorescence spectrum FS′ generated by inverse transformation of unmixing increases. In other words, the reproduction rate of the restored fluorescence spectrum FS′ can be controlled by adjusting the amount of data of the dummy spectral reference SR′. Thus, for example, increase in an amount of data to be transferred from the local environment to the cloud environment can be controlled by adjusting the amount of data of the dummy spectral reference SR′ in accordance with the reproduction rate of the restored fluorescence spectrum FS′ required in the analysis.

Note that as the dummy spectral reference SR′, spectrum information not included in the spectrum references SR used for unmixing can be generated and used. Further, for example, a spectral reference SR corresponding to a fluorescent dye other than the fluorescent dyes used for dyeing the measurement target S may be used as the dummy spectral reference SR′. Still further, the dummy spectral reference SR′ may be any data that can complement spectrum information not included in the spectrum references SR used for unmixing, and, for example, a random number, or the like, can also be used as the dummy spectral reference SR′.

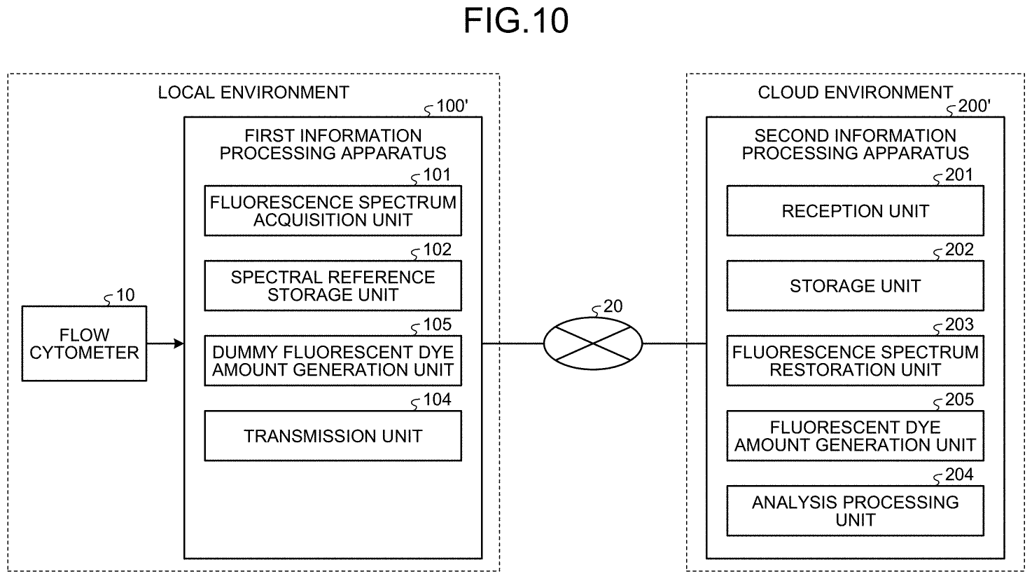

FIG. 10 is a block diagram illustrating a configuration example of an information processing system according to the second embodiment. As illustrated in FIG. 10, in the present embodiment, a first information processing apparatus 100′ provided in the local environment includes a dummy fluorescent dye amount generation unit 105 (an example of a “first processing unit”) instead of the fluorescent dye amount generation unit 103 described above. Furthermore, in the present embodiment, a second information processing apparatus 200′ provided in the cloud environment further includes a fluorescent dye amount generation unit 205 (corresponding to part of the functions of the “second processing unit”).

The dummy fluorescent dye amount generation unit 105 unmixes the fluorescence spectrum FS acquired by the fluorescence spectrum acquisition unit 101 using the spectral reference SR corresponding to each fluorescent dye used for dyeing the measurement target S and the dummy spectral reference SR′ among the spectral references SR stored in the spectral reference storage unit 102 to generate the dummy fluorescent dye amount FC′. As the dummy spectral reference SR′, spectral information not included in the spectral references SR corresponding to the respective fluorescent dyes used for dyeing the measurement target S may be separately generated and used, or a spectral reference SR other than the spectral references SR corresponding to the respective fluorescent dyes used for dyeing the measurement target S may be used among the spectral references SR stored in the spectral reference storage unit 102, or a random number may be used.

In the present embodiment, the transmission unit 104 of the first information processing apparatus 100′ transmits the dummy fluorescent dye amount FC′ generated by the dummy fluorescent dye amount generation unit 105, and the spectral references SR and the dummy spectral reference SR′ used for unmixing at the dummy fluorescent dye amount generation unit 105 to the second information processing apparatus 200′ via the network 20.

The reception unit 201 of the second information processing apparatus 200′ receives the dummy fluorescent dye amount FC′, the spectral references SR, and the dummy spectral reference SR′ transmitted from the first information processing apparatus 100′ via the network 20.

The storage unit 202 stores the dummy fluorescent dye amount FC′, the spectral references SR, and the dummy spectral reference SR′ received by the reception unit 201 from the first information processing apparatus 100′.

The fluorescence spectrum restoration unit 203 uses the dummy fluorescent dye amount FC′, the spectral references SR, and the dummy spectral reference SR′ stored in the storage unit 202 to generate a restored fluorescence spectrum FS′ by performing inverse transformation of unmixing performed when the dummy fluorescent dye amount generation unit 105 of the first information processing apparatus 100′ generates the dummy fluorescent dye amount FC′. In the present embodiment, unmixing and inverse transformation of unmixing are performed using the dummy spectrum reference SR as described above, so that the restored fluorescence spectrum FS′ having high reproducibility can be generated as compared with the first embodiment described above.

The fluorescent dye amount generation unit 205 unmixes the restored fluorescence spectrum FS′ generated by the fluorescence spectrum restoration unit 203 using the spectral references SR stored in the storage unit 202 to generate the fluorescent dye amount FC. The restored fluorescence spectrum FS′ generated by the fluorescence spectrum restoration unit 203 is obtained by restoring the original fluorescence spectrum FS with high reproducibility, and thus, the fluorescent dye amount generation unit 205 can accurately generate the fluorescent dye amount FC by unmixing the restored fluorescence spectrum FS′.

The analysis processing unit 204 uses the fluorescent dye amount FC generated by the fluorescent dye amount generation unit 205 and the restored fluorescence spectrum FS′ generated by the fluorescence spectrum restoration unit 203 to analyze the measurement target S in a similar manner to the first embodiment described above.

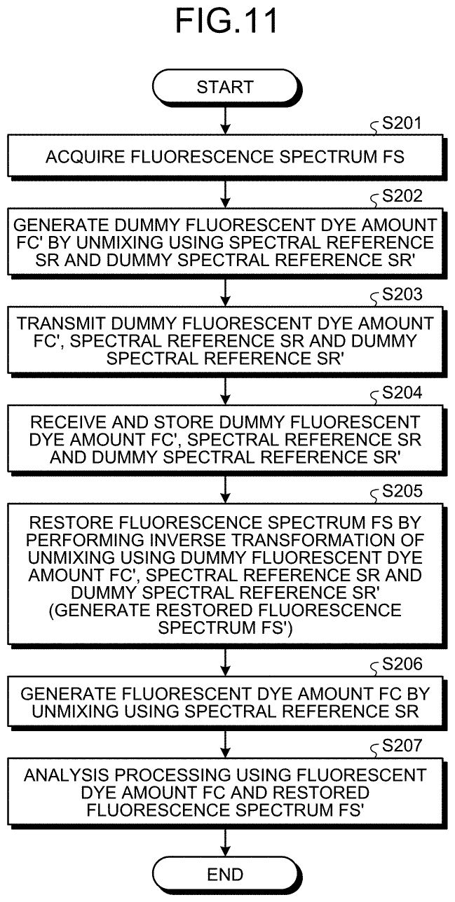

FIG. 11 is a flowchart illustrating flow of a series of processing to be performed in an information processing system according to the second embodiment. Hereinafter, outline of operation of the information processing system according to the present embodiment will be described along the flowchart of FIG. 11.

First, if the fluorescence spectrum FS of the measurement target S dyed using a plurality of fluorescent dyes is measured by the flow cytometer 10, the fluorescence spectrum acquisition unit 101 of the first information processing apparatus 100′ acquires the fluorescence spectrum FS (Step S201).

Next, the dummy fluorescent dye amount generation unit 105 of the first information processing apparatus 100′ unmixes the fluorescence spectrum FS acquired by the fluorescence spectrum acquisition unit 101 in Step S201 using the spectral reference SR corresponding to each fluorescent dye used for dyeing the measurement target S and the dummy spectral reference SR′ among the spectral references SR stored in the spectral reference storage unit 102 to generate the dummy fluorescent dye amount FC′ (Step S202).

Next, the transmission unit 104 of the first information processing apparatus 100′ transmits the dummy fluorescent dye amount FC′ generated by the dummy fluorescent dye amount generation unit 105 in Step S202, and the spectral references SR and the dummy spectral reference SR′ used for unmixing to the second information processing apparatus 200′ via the network 20 (Step S203).

Next, the reception unit 201 of the second information processing apparatus 200′ receives the dummy fluorescent dye amount FC′, the spectral references SR, and the dummy spectral reference SR′ transmitted from the first information processing apparatus 100′ via the network 20 and stores them in the storage unit 202 (Step S204).

Thereafter, the fluorescence spectrum restoration unit 203 of the second information processing apparatus 200′ restores the fluorescence spectrum FS by performing inverse transformation of unmixing performed by the dummy fluorescent dye amount generation unit 105 of the first information processing apparatus 100′ in Step S202, using the dummy fluorescent dye amount FC′ stored in the storage unit 202, the spectral references SR, and the dummy spectral reference SR′ to generate the restored fluorescence spectrum FS′ (Step S205).

Further, the fluorescent dye amount generation unit 205 of the second information processing apparatus 200′ unmixes the restored fluorescence spectrum FS′ generated by the fluorescence spectrum restoration unit 203 in Step S205 using the spectral references SR stored in the storage unit 202 to generate the fluorescent dye amount FC (Step S206).

Then, the analysis processing unit 204 of the second information processing apparatus 200′ performs analysis processing on the measurement target S using the fluorescent dye amount FC generated by the fluorescent dye amount generation unit 205 in Step S206 and the restored fluorescence spectrum FS′ generated by the fluorescence spectrum restoration unit 203 in Step S205 (Step S207), and a series of processing ends.

As described above, according to the present embodiment, unmixing and inverse transformation of unmixing of the fluorescence spectrum FS are performed using the dummy spectral reference SR′ having spectral information not included in the spectral references SR, so that the reproducibility of the restored fluorescence spectrum FS′ generated in the cloud environment can be improved. Further, the reproducibility of the restored fluorescence spectrum FS′ generated in the cloud environment can be controlled by adjusting an amount of data of the dummy spectral reference SR′, so that increase in an amount of data to be transferred from the local environment to the cloud environment can be minimized.

### 4. Third Embodiment

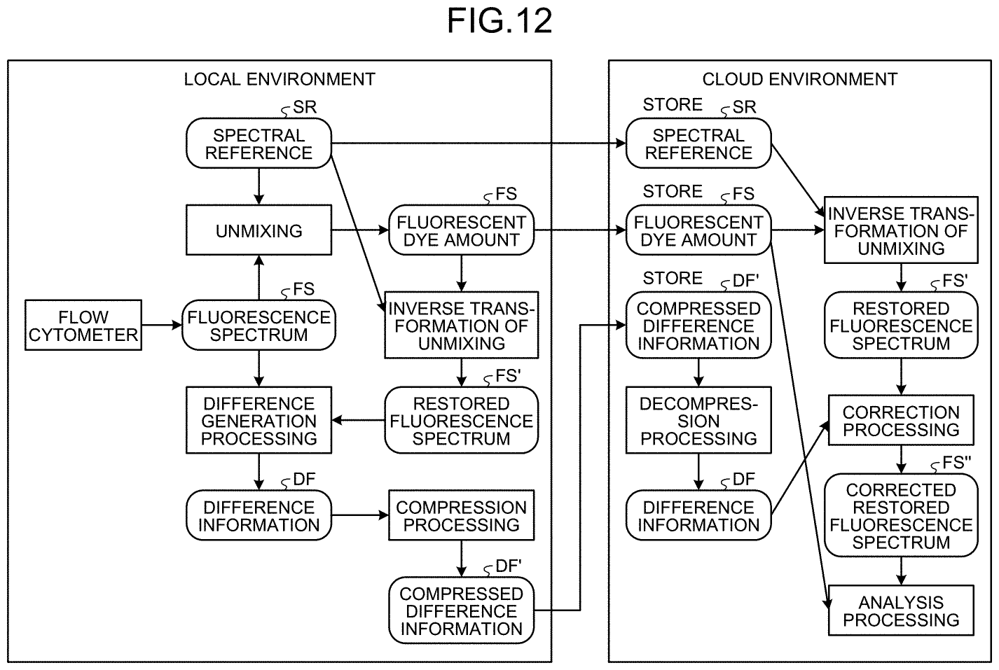

In the present embodiment, another method for increasing the reproduction rate of the fluorescence spectrum FS will be described. An example of data flow of the present embodiment is illustrated in FIG. 12. In the present embodiment, as illustrated in FIG. 12, the restored fluorescence spectrum FS′ is generated by performing inverse transformation of unmixing using the generated fluorescent dye amount FC and the spectral references SR in addition to generating the fluorescent dye amount FC by unmixing of the fluorescence spectrum FS using the spectral references SR in the local environment. Then, difference information DF representing a difference between the restored fluorescence spectrum FS′ generated by inverse transformation of unmixing and the original fluorescence spectrum FS is generated.

The difference information DF has a smaller dynamic range of data, reduces more bits and has a higher compression rate in compression algorithm as compared to the fluorescence spectrum FS. Thus, compressed difference information DF′ obtained by compressing the difference information DF is transferred from the local environment to the cloud environment together with the fluorescent dye amount FC and the spectral references SR and stored in the cloud environment.

In the cloud environment, after the restored fluorescence spectrum FS′ is generated by performing inverse transformation of unmixing using the fluorescent dye amount FC and the spectral references SR, and the restored fluorescence spectrum FS′ is corrected so as to approach the original fluorescence spectrum FS using the difference information DF obtained by decompressing the compressed difference information DF′ to generate the corrected restored fluorescence spectrum FS″ having higher reproducibility than the restored fluorescence spectrum FS′. Then, analysis processing of the measurement target is performed using the fluorescent dye amount FC and the corrected restored fluorescence spectrum FS″.

According to the method of the present embodiment, as an amount of data of the difference information DF increases, the reproduction rate of the corrected fluorescence spectrum FS″ generated by the correction processing increases. In other words, the reproduction rate of the corrected restored fluorescence spectrum FS″ can be controlled by adjusting the amount of data of the difference information DF. It is therefore possible to prevent increase in an amount of data to be transferred from the local environment to the cloud environment by, for example, adjusting the amount of data of the difference information DF in accordance with the reproduction rate of the corrected restored fluorescence spectrum FS″ required in analysis.

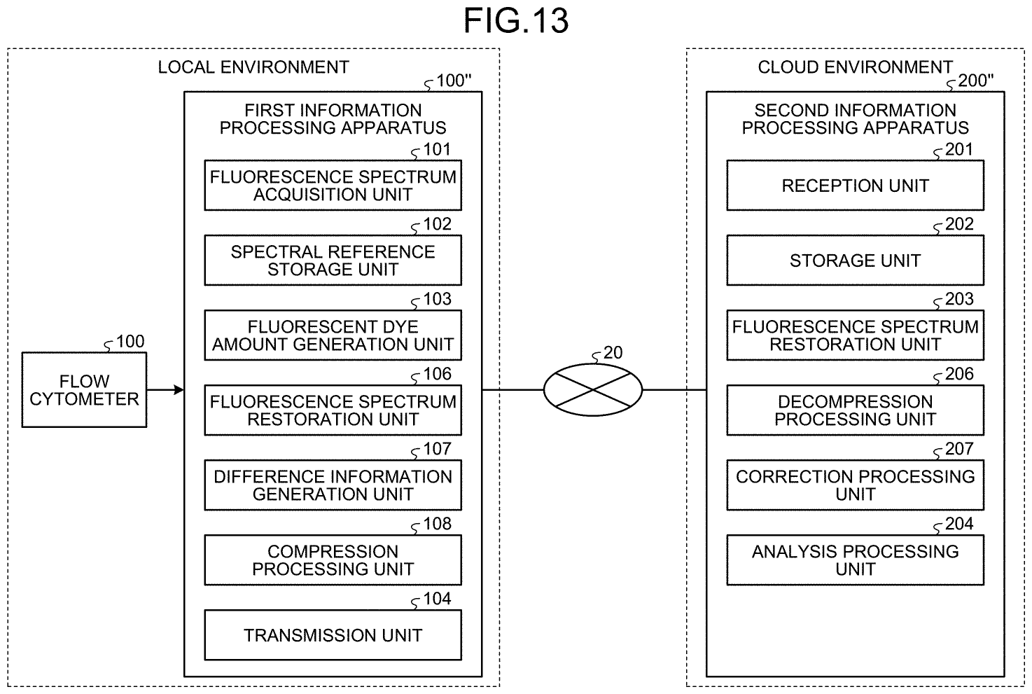

FIG. 13 is a block diagram illustrating a configuration example of an information processing system according to the third embodiment. As illustrated in FIG. 13, in the present embodiment, a first information processing apparatus 100″ provided in the local environment further includes a fluorescence spectrum restoration unit 106 (corresponding to part of the functions of the “first processing unit”), a difference information generation unit 107 (corresponding to part of the functions of the “first processing unit”), and a compression processing unit 108. Further, in the present embodiment, a second information processing apparatus 200″ provided in the cloud environment further includes a decompression processing unit 206 and a correction processing unit 207 (corresponding to part of the functions of the “second processing unit”).

The fluorescence spectrum restoration unit 106 generates a restored fluorescence spectrum FS′ by performing inverse transformation of unmixing using the fluorescent dye amount FC generated by the fluorescent dye amount generation unit 103 and the spectral references SR used for unmixing at the fluorescent dye amount generation unit 103.

The difference information generation unit 107 generates difference information DF representing a difference between the fluorescence spectrum FS and the restored fluorescence spectrum FS′ on the basis of the fluorescence spectrum FS acquired by the fluorescence spectrum acquisition unit 101 and the restored fluorescence spectrum FS′ generated by the fluorescence spectrum restoration unit 106.

The compression processing unit 108 compresses the difference information DF generated by the difference information generation unit 107 using predetermined compression algorithm to generate compressed difference information DF′.

In the present embodiment, the transmission unit 104 of the first information processing apparatus 100″ transmits the compressed difference information DF′ generated by the compression processing unit 108 to the second information processing apparatus 200″ via the network 20, in addition to the fluorescent dye amount FC generated by the fluorescent dye amount generation unit 103 and the spectral references SR used for unmixing at the fluorescent dye amount generation unit 103.

The reception unit 201 of the second information processing apparatus 200″ receives the fluorescent dye amount FC, the spectral references SR, and the compressed difference information DF′ transmitted from the first information processing apparatus 100″ via the network 20.

The storage unit 202 stores the fluorescent dye amount FC, the spectral references SR, and the compressed difference information DF′ received by the reception unit 201 from the first information processing apparatus 100″.

The decompression processing unit 206 decompresses the compressed difference information DF′ stored in the storage unit 202 to restore the difference information DF.

The correction processing unit 207 corrects the restored fluorescence spectrum FS′ generated by the fluorescence spectrum restoration unit 203 so as to approach the original fluorescence spectrum FS using the difference information DF restored by the decompression processing unit 206 to thereby generate the corrected restored fluorescence spectrum FS″ having higher reproducibility than the restored fluorescence spectrum FS′.

The analysis processing unit 204 analyzes the measurement target S in a similar manner to the first embodiment described above using the fluorescent dye amount FC stored in the storage unit 202 and the corrected restored fluorescence spectrum FS″ generated by the correction processing unit 207.

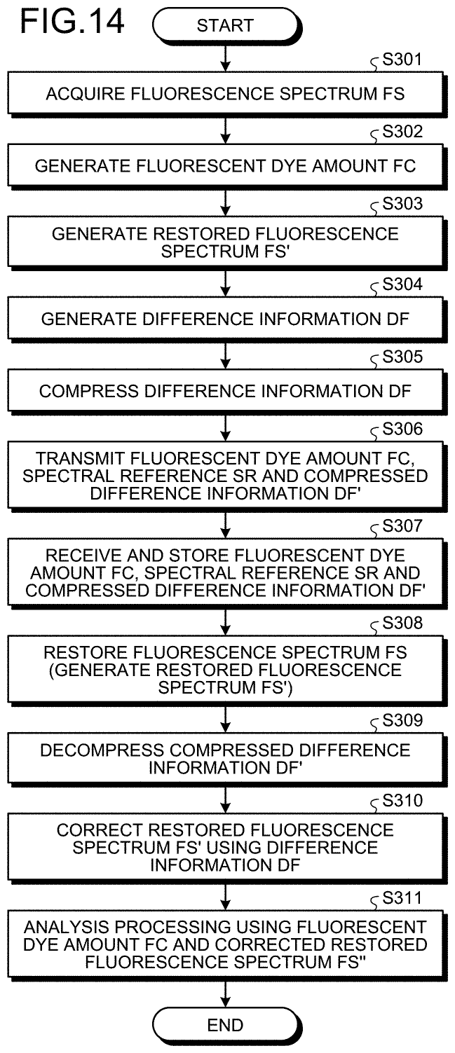

FIG. 14 is a flowchart illustrating flow of a series of processing to be performed in an information processing system according to the third embodiment. Hereinafter, outline of operation of the information processing system according to the present embodiment will be described along the flowchart of FIG. 14.

First, if the fluorescence spectrum FS of the measurement target S dyed using a plurality of fluorescent dyes is measured by the flow cytometer 10, the fluorescence spectrum acquisition unit 101 of the first information processing apparatus 100″ acquires the fluorescence spectrum FS (Step S301).

Next, the fluorescent dye amount generation unit 103 of the first information processing apparatus 100″ unmixes the fluorescence spectrum FS acquired by the fluorescence spectrum acquisition unit 101 in Step S301 using the spectral reference SR corresponding to each fluorescent dye used for dyeing the measurement target S among the spectral references SR stored in the spectral reference storage unit 102 to generate the fluorescent dye amount FC (Step S302).

Then, the fluorescence spectrum restoration unit 106 of the first information processing apparatus 100″ performs inverse transformation of unmixing using the fluorescent dye amount FC generated by the fluorescent dye amount generation unit 103 in Step S302 and the spectral references SR used for unmixing in generating the fluorescent dye amount FC to generate the restored fluorescence spectrum FS′ (Step S303).

Next, the difference information generation unit 107 of the first information processing apparatus 100″ generates difference information DF representing a difference between the fluorescence spectrum FS and the restored fluorescence spectrum FS′ on the basis of the fluorescence spectrum FS acquired by the fluorescence spectrum acquisition unit 101 in Step S301 and the restored fluorescence spectrum FS′ generated by the fluorescence spectrum restoration unit 106 in Step S303 (Step S304).

Then, the compression processing unit 108 of the first information processing apparatus 100″ compresses the difference information DF generated by the difference information generation unit 107 in Step S304 using predetermined compression algorithm to generate compressed difference information DF′ (Step S305). By compressing the difference information DF, an amount of data to be transmitted to the second information processing apparatus 200″ in Step S306 which will be described later can be reduced. However, the difference information DF may be transmitted to the second information processing apparatus 200″ without being compressed. In this case, the processing in Step S305 can be omitted.

Then, the transmission unit 104 of the first information processing apparatus 100″ transmits the fluorescent dye amount FC generated by the fluorescent dye amount generation unit 103 in Step S302, the spectral references SR used for unmixing, and the compressed difference information DF′ generated by the compression processing unit 108 in Step S305 to the second information processing apparatus 200″ via the network 20 (Step S306).

Next, the reception unit 201 of the second information processing apparatus 200″ receives the fluorescent dye amount FC, the spectral references SR, and the compressed difference information DF′ transmitted from the first information processing apparatus 100″ via the network 20 and stores them in the storage unit 202 (Step S307).

Thereafter, the fluorescence spectrum restoration unit 203 of the second information processing apparatus 200″ performs inverse transformation of unmixing performed by the fluorescent dye amount generation unit 103 of the first information processing apparatus 100″ in Step S302, using the fluorescent dye amount FC stored in the storage unit 202 and the spectral references SR to generate the restored fluorescence spectrum FS′ (Step S308).

Then, the decompression processing unit 206 of the second information processing apparatus 200″ decompresses the compressed difference information DF′ stored in the storage unit 202 to restore the difference information DF (Step S309).

Then, the correction processing unit 207 of the second information processing apparatus 200″ corrects the restored fluorescence spectrum FS′ generated by the fluorescence spectrum restoration unit 203 in Step S308 so as to approach the original fluorescence spectrum FS using the difference information DF restored by the decompression processing unit 206 in Step S309 to generate a corrected restored fluorescence spectrum FS″ (Step S310).

Then, the analysis processing unit 204 of the second information processing apparatus 200″ performs analysis processing on the measurement target S using the fluorescent dye amount FC stored in the storage unit 202 and the corrected restored fluorescence spectrum FS″ generated by the correction processing unit 207 in Step S310 (Step S311), and a series of processing ends.

As described above, according to the present embodiment, the difference information DF representing the difference between the original fluorescence spectrum FS and the restored fluorescence spectrum FS′ generated by inverse transformation of unmixing is generated in advance in the local environment and transferred to the cloud environment, and the restored fluorescence spectrum FS′ generated by inverse transformation of unmixing in the cloud environment is corrected using the difference information DF, so that it is possible to perform analysis processing using the corrected restored fluorescence spectrum FS″ having higher reproducibility than the restored fluorescence spectrum FS′. Further, the reproducibility of the corrected restored fluorescence spectrum FS″ generated in the cloud environment can be controlled by adjusting an amount of data of the difference information DF, so that increase in an amount of data to be transferred from the local environment to the cloud environment can be minimized.

### 5. Fourth Embodiment

While the flow cytometer 10 described in each of the embodiments described above measures the fluorescence spectrum FS to be used for analysis of the measurement target S, there is also an apparatus having a function of sorting one which emits specific fluorescence from the measurement target S by controlling a movement destination of the measurement target S such as cells passing through the flow cell 12 on the basis of the measured fluorescence spectrum FS. The flow cytometer 10′ having such a sorting function is called a sorter (cell sorter).

In the present embodiment, an application example to an information processing system using the flow cytometer 10′ having such a sorting function will be described. In the flow cytometer 10′ having a sorting function, it is necessary to instantaneously determine whether the measurement target S is a sorting target on the basis of the fluorescence spectrum FS measured by irradiating the measurement target S passing through the flow cell 12 with light and control the movement destination of the measurement target S. Here, by constructing a learning model through machine learning using the fluorescence spectrum FS measured from the measurement target S which is a sorting target as learning data and performing determination using this learning model, it is possible to instantaneously perform determination on the basis of the fluorescence spectrum FS. In the present embodiment, it is considered that such a learning model is constructed in the cloud environment.

In the present embodiment, first, the fluorescence spectrum FS for constructing the learning model is measured by the flow cytometer 10′. The measured fluorescence spectrum FS is not transferred from the local environment to the cloud environment, but is restored by being subjected to inverse transformation of unmixing in the cloud environment in a similar manner to the above-described embodiments. Then, in a similar manner to each of the above-described embodiments, analysis processing such as clustering processing is performed on the measurement target S in the cloud environment, and a result of the analysis processing is presented to the user.

Here, if the measurement target S which is the sorting target is specified by the user who refers to the presented result of the analysis processing, machine learning is performed using the restored fluorescence spectrum FS′ (or the corrected restored fluorescence spectrum FS″) corresponding to the specified measurement target S which is the sorting target as learning data, so that a learning model is constructed. Then, the learning model constructed in the cloud environment is transferred to the local environment.

Thereafter, in the local environment, the sorting target is determined using the learning model transferred from the cloud environment. In other words, if the fluorescence spectrum FS is measured by the flow cytometer 10′, whether or not the measurement target S having this fluorescence spectrum is a sorting target is determined on the basis of the learning model. Then, the movement destination of the sorting target S is controlled on the basis of a result of the determination, and the measurement target S designated as the sorting target is sorted.

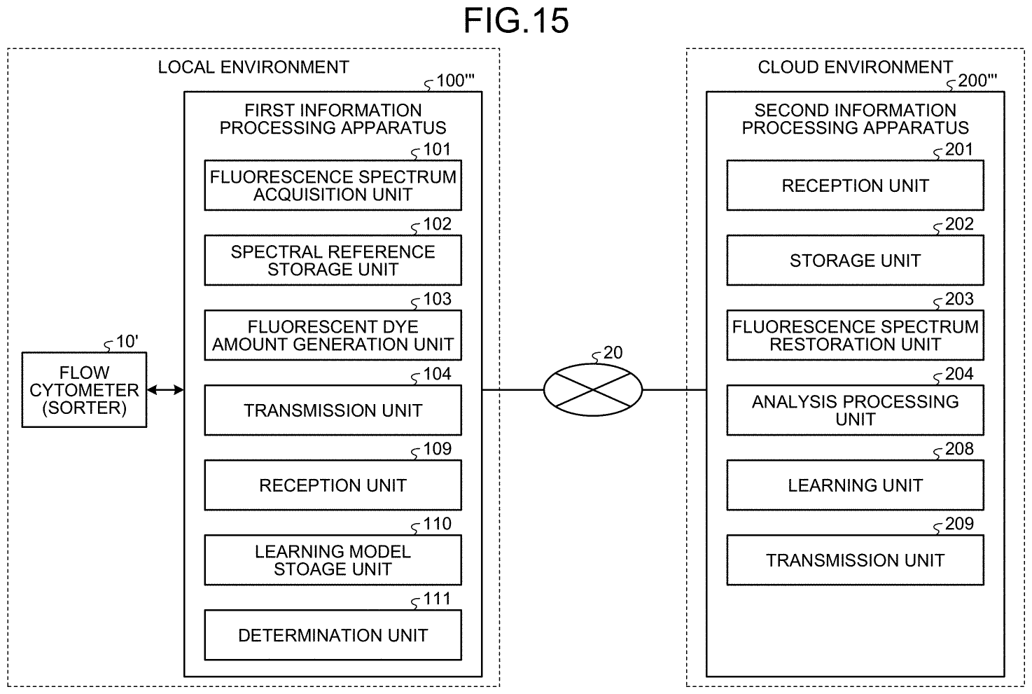

FIG. 15 is a block diagram illustrating a configuration example of an information processing system according to a fourth embodiment. As illustrated in FIG. 15, in the present embodiment, a second information processing apparatus 200′″ provided in the cloud environment further includes a learning unit 208 and a transmission unit 209 (corresponding to a “learning model transmission unit”). Further, in the present embodiment, a first information processing apparatus 100′″ provided in the local environment further includes a reception unit 109 (corresponding to a “learning model reception unit”), a learning model storage unit 110, and a determination unit 111. Note that while FIG. 15 illustrates a configuration example in which a configuration specific to the present embodiment is added to the information processing system according to the above-described first embodiment, a base information processing system may be the information processing system according to the above-described second embodiment or the information processing system according to the above-described third embodiment.

If a specific measurement target S is designated as the sorting target by the user who refers to the analysis result of the analysis processing unit 204, the learning unit 208 of the second information processing apparatus 200′″ performs machine learning using the restored fluorescence spectrum FS′ corresponding to the measurement target S designated as the sorting target as learning data to thereby construct a learning model for determining whether or not the measurement target S is the sorting target on the basis of the fluorescence spectrum FS.

Algorithm of machine learning to be performed by the learning unit 208 is supervised learning using the restored fluorescence spectrum FS′ corresponding to the measurement target S designated as the sorting target as learning data. For example, the learning unit 208 may construct the learning model using machine learning algorithm such as random forest, support vector machine, or deep learning.

Note that the learning unit 208 may determine whether or not a learning model capable of sufficiently determining the sorting target has been constructed and may notify the user of the determination result. For example, in a case where the number of the learned restored fluorescence spectrum FS′ of the measurement target S or a ratio of the learned restored fluorescence spectrum FS′ to the whole exceeds a threshold value, the learning unit 208 may notify the user that a learning model capable of sufficiently determining the sorting target has been constructed.

Alternatively, in a case where a correct answer rate of the learning model exceeds the threshold value, the learning unit 208 may notify the user that the learning model capable of sufficiently determining the sorting target has been constructed. The correct answer rate of the learning model can be determined by, for example, N-fold-cross validation. Specifically, the correct answer rate of the constructed learning model can be determined by dividing the whole of the learning data into N, performing learning with the learning data included in the N−1 divided portions to construct a learning model and then performing determination using the learning data included in the remaining one divided portion.

The transmission unit 209 of the second information processing apparatus 200′″ transmits the learning model constructed by the learning unit 208 to the first information processing apparatus 100′″ via the network 20.

The reception unit 109 of the first information processing apparatus 100′″ receives the learning model transmitted from the second information processing apparatus 200′″ via the network 20.

The learning model storage unit 110 stores the learning model received by the reception unit 109 from the second information processing apparatus 200′″.

If the fluorescence spectrum FS of the measurement target S is measured by the flow cytometer 10′ after the learning model storage unit 110 stores the learning model, the determination unit 111 determines whether or not the measurement target S having the fluorescence spectrum FS is a sorting target on the basis of the learning model stored in the learning model storage unit 110. Then, in a case where the measurement target S is determined as the sorting target, the determination unit 111 outputs an instruction to the flow cytometer 10′ to sort the measurement target S. Further, in a case where the flow cytometer 10′ is capable of separately sorting a plurality of groups of the measurement target S, the determination unit 111 may instruct the flow cytometer 10′ not only whether or not the measurement target S is a sorting target but also to which collection unit the measurement target S is to be collected.

Note that the learning model storage unit 110 and the determination unit 111 may be provided at the flow cytometer 10′. In addition, the constructed learning model may be implemented in a logic circuit such as an FPGA circuit provided at the flow cytometer 10′. For example, the determination unit 111 may be provided at the flow cytometer 10′, and logic which executes the learning model designed and constructed on the basis of the type of the determination unit 111 may be implemented at the FPGA circuit provided at the flow cytometer 10′.

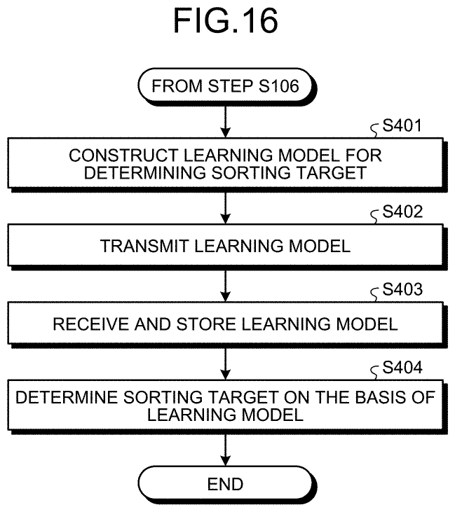

FIG. 16 is a flowchart illustrating characteristic processing to be performed in the information processing system according to the fourth embodiment and illustrates, for example, flow of processing to be performed subsequent to the analysis processing in Step S106 illustrated in the flowchart of FIG. 8. Note that, in the analysis processing, it is assumed that a result of processing such as clustering processing is presented to the user, and the sorting target is designated by the user who refers to the presented result of the analysis processing.

If the sorting target is designated by the user, the learning unit 208 of the second information processing apparatus 200′″ performs machine learning using the restored fluorescence spectrum FS′ corresponding to the measurement target S designated as the sorting target as learning data and constructs a learning model for determining the sorting target (Step S401).

Next, the transmission unit 209 of the second information processing apparatus 200′″ transmits the learning model constructed by the learning unit 208 in Step S401 to the first information processing apparatus 100′″ via the network 20 (Step S402).

Next, the reception unit 109 of the first information processing apparatus 100′″ receives the learning model transmitted from the second information processing apparatus 200′″ via the network 20 and stores the learning model in the learning model storage unit 110 (Step S403).

Thereafter, if the fluorescence spectrum FS of the measurement target S is measured by the flow cytometer 10′, the determination unit 111 of the first information processing apparatus 100′ determines whether or not the measurement target S is a sorting target on the basis of the learning model stored in the learning model storage unit 110 (Step S404) and issues an instruction to the flow cytometer 10′. As a result, the measurement target S specified as the sorting target by the user can be appropriately sorted by the flow cytometer 10′.

As described above, according to the present embodiment, construction of a learning model which requires heavy processing load is performed in the cloud environment, and the sorting target is determined using the learning model constructed in the cloud environment to cause the flow cytometer 10′ to appropriately perform operation, so that user-friendliness is improved. Further, it is not necessary to transfer the fluorescence spectrum FS having a large data size from the first information processing apparatus 100′″ in the local environment to the second information processing apparatus 200′″ in the cloud environment to construct the learning model in the cloud environment, so that it is possible to reduce an amount of data to be transferred from the local environment to the cloud environment, and reduce an amount of data stored in the cloud environment, so that it is possible to reduce storage cost.

### 6. Fifth Embodiment

Note that while in each of the above-described embodiments, the fluorescence spectrum FS of the measurement target S is measured by the flow cytometer 10 (10′), a mechanism of the present disclosure can also be effectively applied, for example, to a case of using a fluorescence imaging apparatus that measures the fluorescence spectrum FS using an imaging element (two-dimensional image sensor). In the present embodiment, an example of application to an information processing system using such a fluorescence imaging apparatus will be described.

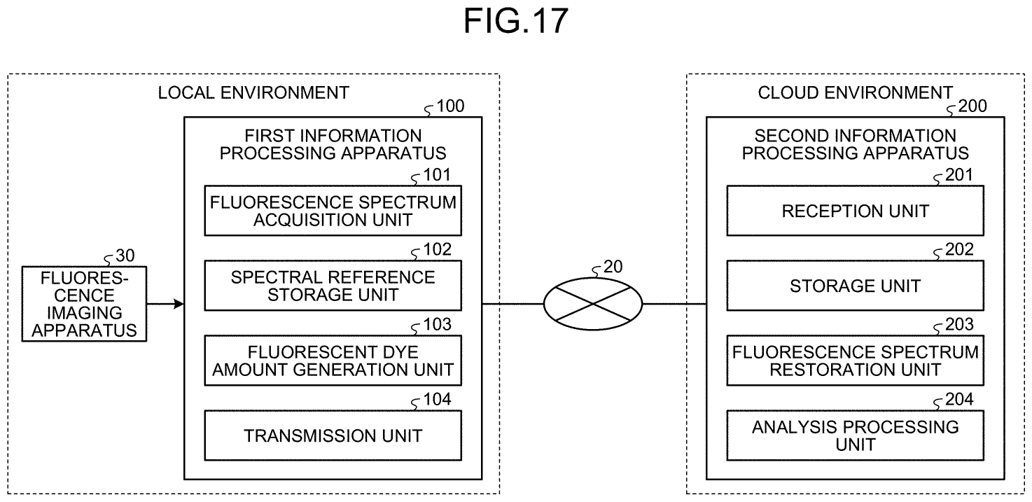

FIG. 17 is a block diagram illustrating a configuration example of an information processing system according to a fifth embodiment. As illustrated in FIG. 17, in the present embodiment, a fluorescence imaging apparatus 30 is provided in the local environment in place of the flow cytometer 10 (10′). Note that a basic configuration of the information processing system is similar to the configuration in the first embodiment illustrated in FIG. 5.

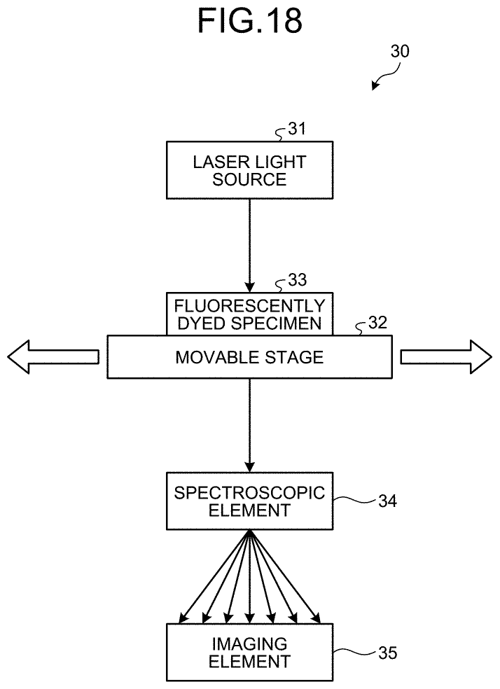

FIG. 18 illustrates a schematic configuration example of the fluorescence imaging apparatus 30. For example, as illustrated in FIG. 18, the fluorescence imaging apparatus 30 includes a laser light source 31, a movable stage 32, a spectroscopic element 34, and an imaging element 35.

The laser light source 31 emits laser light having a wavelength capable of exciting fluorescent dyes used for dyeing the measurement target S. As the laser light source 31, for example, a semiconductor laser light source that emits laser light having a predetermined wavelength can be used.

A fluorescently dyed specimen 33 is placed on the movable stage 32. The movable stage 32 moves in a horizontal direction so that the laser light emitted from the laser light source 31 scans the fluorescently dyed specimen 33 in two dimensions.

The fluorescently dyed specimen 33 is, for example, a sample collected from a human body or a sample prepared from a tissue sample for the purpose of pathological diagnosis, or the like, and dyed using a plurality of fluorescent dyes. The fluorescently dyed specimen 33 includes a large number of measurement targets S such as cells constituting the collected tissue. By moving the movable stage 32 so that the laser light emitted from the laser light source 31 scans the fluorescently dyed specimen 33 in two dimensions, a large number of measurement targets S included in the fluorescently dyed specimen 33 can be sequentially irradiated with the laser light.

The spectroscopic element 34 is an optical element that disperses fluorescence emitted by irradiating the measurement targets S included in the fluorescently dyed specimen 33 with laser light into a spectrum of a continuous wavelength. As the spectroscopic element 34, for example, a prism, a grating, or the like, can be used.

The imaging element 35 is a two-dimensional image sensor in which light receiving elements such as a charge coupled device (CCD) sensor and a complementary metal oxide semiconductor (CMOS) sensor are arranged in two dimensions. The imaging element 35 receives the fluorescence emitted by irradiating the measurement targets S included in the fluorescently dyed specimen 33 with the laser light and dispersed by the spectroscopic element 34, by the respective light receiving elements arranged in two dimensions and outputs an image signal. The fluorescence emitted from the measurement targets S by being irradiated with the laser light is dispersed into a continuous spectrum by the spectroscopic element 13, and thus, the imaging element 35 outputs an image signal corresponding to the fluorescence intensity in a wavelength region different for each region.

In the fluorescence imaging apparatus 30 configured as described above, the fluorescence emitted by irradiating the measurement targets S included in the fluorescently dyed specimen 33 with the laser light is dispersed into a continuous spectrum by the spectroscopic element 34 and detected by the respective light receiving elements of the imaging element 35. Thus, the fluorescence spectrum FS of the measurement target S can be measured using the image signal output from the imaging element 35, in a similar manner to the flow cytometer 10 (10′).

In the information processing system according to the present embodiment, the fluorescence spectrum acquisition unit 101 of the first information processing apparatus 100 provided in the local environment acquires the fluorescence spectrum FS of the measurement target S measured by the fluorescence imaging apparatus 30. The subsequent processing is similar to that of the first embodiment described above, and thus, the description thereof will be omitted. Note that while FIG. 17 illustrates an example where the flow cytometer 10 in the information processing system according to the above-described first embodiment is replaced with the fluorescence imaging apparatus 30, a base information processing system may be the information processing system according to the above-described second embodiment or the information processing system according to the above-described third embodiment.

As described above, even in a case where the fluorescence spectrum FS of the measurement target S is measured by the fluorescence imaging apparatus 30, by applying the mechanism of the present disclosure, it is possible to reduce an amount of data to be transferred from the local environment to the cloud environment and reduce an amount of data stored in the cloud environment, so that it is possible to reduce storage cost.

### 7. Hardware Configuration Example

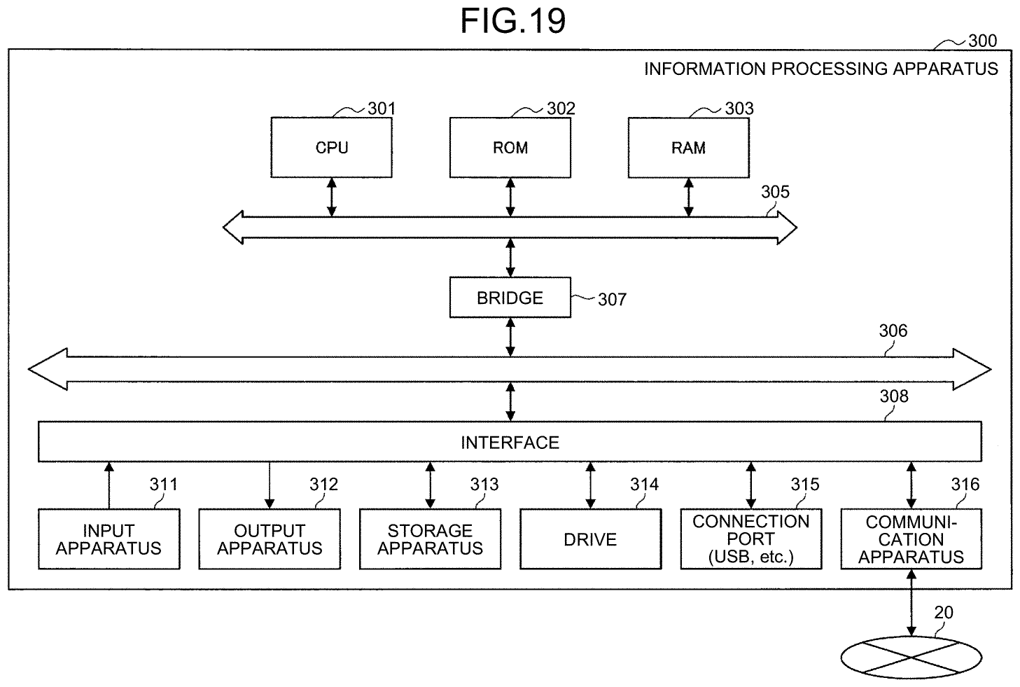

Subsequently, an example of a hardware configuration of the first information processing apparatus 100 and the second information processing apparatus 200 (hereinafter, these will be collectively referred to as an “information processing apparatus 300”) will be described with reference to FIG. 19. FIG. 19 is a block diagram illustrating an example of a hardware configuration of the information processing apparatus 300.

As illustrated in FIG. 19, the information processing apparatus 300 includes a central processing unit (CPU) 301, a read only memory (ROM) 302, a random access memory (RAM) 303, a host bus 305, a bridge 307, an external bus 306, an interface 308, an input apparatus 311, an output apparatus 312, a storage apparatus 313, a drive 314, a connection port 315, and a communication apparatus 316. The information processing apparatus 300 may include a processing circuit such as an electric circuit, a DSP, or an ASIC in place of or in addition to the CPU 301.

The CPU 301 functions as an arithmetic processing unit and a control unit, and controls overall operation in the information processing apparatus 300 in accordance with various programs. Furthermore, the CPU 301 may be a microprocessor. The ROM 302 stores programs, operation parameters, and the like, used by the CPU 301. The RAM 303 temporarily stores programs to be used in execution of the CPU 301 and parameters that vary as appropriate in this execution, and the like. The CPU 301 may implement functions of, for example, the fluorescence spectrum acquisition unit 101 and the fluorescent dye amount generation unit 103 in the first information processing apparatus 100 described above. In addition, the CPU 301 may implement functions of, for example, the fluorescence spectrum restoration unit 203 and the analysis processing unit in the second information processing apparatus 200 described above.

The CPU 301, the ROM 302, and the RAM 303 are mutually connected by the host bus 305 including a CPU bus, and the like. The host bus 305 is connected to the external bus 306 such as a peripheral component interconnect/interface (PCI) bus via the bridge 307. Note that the host bus 305, the bridge 307, and the external bus 306 are not necessarily separated, and these functions may be implemented in one bus.

The input apparatus 311 is an apparatus such as a mouse, a keyboard, a touch panel, buttons, a microphone, a switch, and a lever to which information is input by the user. Alternatively, the input apparatus 311 may be, for example, a remote control apparatus using infrared rays or other radio waves or may be external connection equipment such as a mobile phone or a PDA corresponding to operation of the information processing apparatus 300. Further, the input apparatus 311 may include, for example, an input control circuit that generates an input signal on the basis of information input by the user using the input means described above.

The output apparatus 312 is an apparatus capable of visually or audibly notifying the user of information. The output apparatus 312 may be, for example, a display apparatus such as a cathode ray tube (CRT) display apparatus, a liquid crystal display apparatus, a plasma display apparatus, an electro luminescence (EL) display apparatus, a laser projector, a light emitting diode (LED) projector, or a lamp, or may be an audio output apparatus such as a speaker or a headphone.

The output apparatus 312 may output, for example, results obtained by various kinds of processing by the information processing apparatus 300. Specifically, the output apparatus 312 may visually display the results obtained by various kinds of processing by the information processing apparatus 300 in various formats such as text, image, table, or graph. Alternatively, the output apparatus 312 may convert an audio signal such as audio data or acoustic data into an analog signal and aurally output the analog signal. The input apparatus 311 and the output apparatus 312 may execute, for example, the functions of the interface unit 309.

The storage apparatus 313 is an apparatus for data storage formed as an example of a storage unit of the information processing apparatus 300. The storage apparatus 313 may be implemented by, for example, a magnetic storage apparatus such as a hard disk drive (HDD), a semiconductor storage apparatus such as a solid state drive (SSD), an optical storage apparatus, a magneto-optical storage apparatus, or the like. For example, the storage apparatus 313 may include a storage medium, a recording apparatus that records data in the storage medium, a reading apparatus that reads data from the storage medium, a deletion apparatus that deletes data recorded in the storage medium, and the like. The storage apparatus 313 may store programs to be executed by the CPU 301, various kinds of data, various kinds of data acquired from outside, and the like. The storage apparatus 313 may implement functions of, for example, the spectral reference storage unit 102 in the first information processing apparatus 100 described above.

The drive 314, which is a reader/writer for a storage medium, is built in or externally attached to the information processing apparatus 300. The drive 314 reads out information recorded in a removable storage medium such as a mounted magnetic disk, optical disk, magneto-optical disk, or semiconductor memory and outputs the information to the RAM 303. Further, the drive 314 can also write information to a removable storage medium.

The connection port 315 is an interface connected to external equipment. The connection port 315 is a connection port capable of transmitting data to external equipment and may be, for example, a universal serial bus (USB).

The communication apparatus 316 is, for example, an interface formed by a communication device, or the like, for connecting to the network 20. The communication apparatus 316 may be, for example, a communication card for wired or wireless local area network (LAN), long term evolution (LTE), Bluetooth (registered trademark), wireless USB (WUSB), or the like. Further, the communication apparatus 316 may be a router for optical communication, a router for asymmetric digital subscriber line (ADSL), a modem for various kinds of communication, or the like. For example, the communication apparatus 316 can transmit and receive signals, and the like, to and from the Internet or other communication equipment in accordance with predetermined protocol such as TCP/IP. The communication apparatus 316 may implement, for example, the functions of, for example, the transmission unit 104 in the first information processing apparatus 100 described above. Further, the communication apparatus 316 may implement the functions of, for example, the reception unit 201 in the second information processing apparatus 200 described above.

Note that it is also possible to create a computer program causing hardware such as the CPU 301, the ROM 302, and the RAM 303 built in the information processing apparatus 300 to exert functions equivalent to the functions of the components of the first information processing apparatus 100 and the second information processing apparatus 200 described above. Further, a storage medium in which the computer program is stored can also be provided.

### 8. Supplementary Explanation

As described above, the favorable embodiments of the present disclosure have been described in detail with reference to the accompanying drawings, but the technical scope of the present disclosure is not limited to such examples. It is obvious that persons having ordinary knowledge in the technical field of the present disclosure can conceive various changes and alterations within the scope of the technical idea described in the claims, and it is naturally understood that these changes and alterations belong to the technical scope of the present disclosure.

Furthermore, the effects described in the present specification are merely illustrative or exemplary and are not restrictive. That is, the technology according to the present disclosure can exhibit other effects obvious to those skilled in the art from the description of the present specification in addition to or in place of the above-described effects.

Note that the following configurations also belong to the technical scope of the present disclosure.

(1)

An information processing system comprising: a first information processing apparatus; and a second information processing apparatus,

the first information processing apparatus including:

a first processing unit configured to generate compressed data by irradiating a measurement target dyed with a plurality of fluorescent dyes with light and performing compression processing on measurement data measured by the irradiation, using reference data for each of the fluorescent dyes used for dyeing the measurement target; and

a transmission unit configured to transmit the compressed data to the second information processing apparatus,

the second information processing apparatus including: a second processing unit configured to generate restored data by performing restoration processing using the reference data and the compressed data received from the first information processing apparatus.

(2)

The information processing system according to (1), wherein the measurement target is a biogenic particle including at least one of a cell, a tissue, a microorganism, or a bio-related particle.

(3)

The information processing system according to (1) or (2), wherein the first information processing apparatus and the second information processing apparatus are connected so as to be able to perform communication with each other via a predetermined network.

(4)

The information processing system according to any one of (1) to (3), wherein the compression processing includes at least one of linear processing or nonlinear processing.

(5)

The information processing system according to any one of (1) to (4), wherein the compression processing includes at least one of dimension compression processing, clustering processing or grouping processing.

(6)

The information processing system according to any one of (1) to (5), wherein the compressed data is a fluorescent dye amount representing a measurement result for each of the fluorescent dyes used for dyeing the measurement target.

(7)

The information processing system according to any one of (1) to (6), wherein the restoration processing is inverse transformation processing of the compressed data.

(8)

The information processing system according to any one of (1) to (7),

wherein the first processing unit performs the compression processing on the measurement data using dummy reference data in addition to the reference data to generate dummy compressed data in which dummy data is added to the compressed data,

the transmission unit transmits the dummy compressed data and the dummy reference data to the second information processing apparatus, and

the second processing unit generates the restored data by performing the restoration processing using the reference data, the dummy compressed data, and the dummy reference data.

(9)

The information processing system according to any one of (1) to (8),

wherein the first processing unit further restores the measurement data by performing inverse transformation of unmixing using the compressed data and the reference data and generates difference information representing a difference between restored measurement data that is the restored measurement data and the measurement data,

the transmission unit further transmits the difference information to the second information processing apparatus,

the second information processing apparatus further receives the difference information from the first information processing apparatus, and

the second processing unit further corrects the restored measurement data on a basis of the difference information.

(10)

The information processing system according to any one of (1) to (9),

wherein the second information processing apparatus further includes:

an analysis processing unit configured to analyze the measurement target using the compressed data and restored measurement data that is the measurement data obtained by restoring the compressed data.

(11)

The information processing system according to (10),

wherein the second information processing apparatus further includes:

a learning unit configured to construct a learning model for determining a sorting target by performing machine learning using the measurement data corresponding to the sorting target specified on a basis of an analysis result by the analysis processing unit; and

a learning model transmission unit configured to transmit the learning model to the first information processing apparatus, and

the first information processing apparatus further includes:

a learning model reception unit configured to receive the learning model from the second information processing apparatus; and

a determination unit configured to determine the sorting target on a basis of the learning model.

(12)

The information processing system according to any one of (1) to (11),

wherein the measurement data is a fluorescence signal obtained by measuring fluorescence emitted from the measurement target.

(13)

The information processing system according to any one of (1) to (11),

wherein the measurement data is image data obtained by imaging the measurement target.

(14)

An information processing apparatus comprising:

a first processing unit configured to generate compressed data by performing compression processing on measurement data measured by irradiating a measurement target dyed using a plurality of fluorescent dyes with light, using reference data for each of the fluorescent dyes used for dyeing the measurement target; and

a second processing unit configured to generate restored data by performing restoration processing using the reference data and the compressed data.

### REFERENCE SIGNS LIST

10, 10′ FLOW CYTOMETER
20 NETWORK
30 FLUORESCENCE IMAGING APPARATUS
100, 100′, 100″, 100′″ FIRST INFORMATION PROCESSING APPARATUS
101 FLUORESCENCE SPECTRUM ACQUISITION UNIT
102 SPECTRAL REFERENCE STORAGE UNIT
103 FLUORESCENT DYE AMOUNT GENERATION UNIT (FIRST PROCESSING UNIT)
104 TRANSMISSION UNIT
105 DUMMY FLUORESCENT DYE AMOUNT GENERATION UNIT (FIRST PROCESSING UNIT)
106 FLUORESCENCE SPECTRUM RESTORATION UNIT (FIRST PROCESSING UNIT)
107 DIFFERENCE INFORMATION GENERATION UNIT (FIRST PROCESSING UNIT)
108 COMPRESSION PROCESSING UNIT
109 RECEPTION UNIT
110 LEARNING MODEL STORAGE UNIT
111 DETERMINATION UNIT
200, 200′, 200″, 200′″ SECOND INFORMATION PROCESSING APPARATUS
201 RECEPTION UNIT
202 STORAGE UNIT
203 FLUORESCENCE SPECTRUM RESTORATION UNIT (SECOND PROCESSING UNIT)
204 ANALYSIS PROCESSING UNIT
205 FLUORESCENT DYE AMOUNT GENERATION UNIT (SECOND PROCESSING UNIT)
206 DECOMPRESSION PROCESSING UNIT
207 CORRECTION PROCESSING UNIT (SECOND PROCESSING UNIT)
208 LEARNING UNIT
209 TRANSMISSION UNIT
FS FLUORESCENCE SPECTRUM (MEASUREMENT DATA)
FS′ RESTORED FLUORESCENCE SPECTRUM (RESTORED MEASUREMENT DATA)
FS″ CORRECTED RESTORED FLUORESCENCE SPECTRUM
SR SPECTRAL REFERENCE (REFERENCE DATA)
SR′ DUMMY SPECTRAL REFERENCE (DUMMY REFERENCE DATA)
FC FLUORESCENT DYE AMOUNT (COMPRESSED DATA)
FC′ DUMMY FLUORESCENT DYE AMOUNT (DUMMY MEASUREMENT DATA)
DF DIFFERENCE INFORMATION
DF′ COMPRESSED DIFFERENCE INFORMATION

1. An information processing system comprising: a first information processing apparatus; and a second information processing apparatus,
the first information processing apparatus including:
a first processing unit configured to generate compressed data by irradiating a measurement target dyed with a plurality of fluorescent dyes with light and performing compression processing on measurement data measured by the irradiation, using reference data for each of the fluorescent dyes used for dyeing the measurement target; and
a transmission unit configured to transmit the compressed data to the second information processing apparatus,
the second information processing apparatus including: a second processing unit configured to generate restored data by performing restoration processing using the reference data and the compressed data received from the first information processing apparatus.

2. The information processing system according to claim 1, wherein the measurement target is a biogenic particle including at least one of a cell, a tissue, a microorganism, or a bio-related particle.

3. The information processing system according to claim 1, wherein the first information processing apparatus and the second information processing apparatus are connected so as to be able to perform communication with each other via a predetermined network.

4. The information processing system according to claim 1, wherein the compression processing includes at least one of linear processing or nonlinear processing.

5. The information processing system according to claim 1, wherein the compression processing includes at least one of dimension compression processing, clustering processing or grouping processing.

6. The information processing system according to claim 1, wherein the compressed data is a fluorescent dye amount representing a measurement result for each of the fluorescent dyes used for dyeing the measurement target.

7. The information processing system according to claim 1, wherein the restoration processing is inverse transformation processing of the compressed data.

8. The information processing system according to claim 1,
wherein the first processing unit performs the compression processing on the measurement data using dummy reference data in addition to the reference data to generate dummy compressed data in which dummy data is added to the compressed data,
the transmission unit transmits the dummy compressed data and the dummy reference data to the second information processing apparatus, and
the second processing unit generates the restored data by performing the restoration processing using the reference data, the dummy compressed data, and the dummy reference data.

9. The information processing system according to claim 1,
wherein the first processing unit further restores the measurement data by performing inverse transformation of unmixing using the compressed data and the reference data and generates difference information representing a difference between restored measurement data that is the restored measurement data and the measurement data,
the transmission unit further transmits the difference information to the second information processing apparatus,
the second information processing apparatus further receives the difference information from the first information processing apparatus, and
the second processing unit further corrects the restored measurement data on a basis of the difference information.

10. The information processing system according to claim 1,
wherein the second information processing apparatus further includes:
an analysis processing unit configured to analyze the measurement target using the compressed data and restored measurement data that is the measurement data obtained by restoring the compressed data.

11. The information processing system according to claim 10,
wherein the second information processing apparatus further includes:
a learning unit configured to construct a learning model for determining a sorting target by performing machine learning using the measurement data corresponding to the sorting target specified on a basis of an analysis result by the analysis processing unit; and
a learning model transmission unit configured to transmit the learning model to the first information processing apparatus, and
the first information processing apparatus further includes:
a learning model reception unit configured to receive the learning model from the second information processing apparatus; and
a determination unit configured to determine the sorting target on a basis of the learning model.

12. The information processing system according to claim 1,
wherein the measurement data is a fluorescence signal obtained by measuring fluorescence emitted from the measurement target.

13. The information processing system according to claim 1,
wherein the measurement data is image data obtained by imaging the measurement target.

14. An information processing apparatus comprising:
a first processing unit configured to generate compressed data by performing compression processing on measurement data measured by irradiating a measurement target dyed using a plurality of fluorescent dyes with light, using reference data for each of the fluorescent dyes used for dyeing the measurement target; and
a second processing unit configured to generate restored data by performing restoration processing using the reference data and the compressed data.
